(19) United States
(12) Patent Application Publication
(10) Pub. No.: US 2021/0269806 A1
(43) Pub. Date: Sep. 2, 2021

(54) IL-1 BETA TARGETING SPHERICAL NUCLEIC ACIDS

(71) Applicant: Exicure, Inc., Chicago, IL (US)

(72) Inventor: Bart Anderson, Morton Grove, IL (US)

(73) Assignee: Exicure, Inc., Chicago, IL (US)

(21) Appl. No.: 17/253,102

(22) PCT Filed: Jun. 20, 2019
(86) PCT No.: PCT/US2019/038266

§ 371 Date: Dec. 16, 2020

Related U.S. Application Data
(60) Provisional application No. 62/687,755, filed on Jun. 20, 2018.

(51) Int. Cl. C12N 15/113; A61K 9/127; A61K 47/54

(52) U.S. Cl. CPC C12N 15/1136; A61K 9/1271; A61K 47/554; C12N 2310/3515; C12N 2310/321; C12N 2310/11; C12N 2310/315

(57) ABSTRACT

The invention in some aspects relates to antisense inhibitors of IL1B and related methods of use. The antisense inhibitors may be formulated as a nanoparticle or other type of three dimensional presentation format.

### RELATED APPLICATIONS

This application claims the benefit of U.S. Provisional Application No. 62/687,755 filed on Jun. 20, 2018, the entire contents of which are incorporated by reference herein.

### BACKGROUND

Inflammation is a physiological response to many stimuli that results in the release of chemical signals by the immune system. In order to communicate and organize this response, cells rely on chemical signaling by many types of cytokines, including but not limited to chemokines, interleukins (ILs), tumor necrosis factor (TNF), and interferons (IFNs). ILs can result in both inflammatory and anti-inflammatory responses. When this system does not operate properly, positive feedback loops can form that progressively intensify the immune response to an inappropriately high level.

Interleukin 1 (IL-1) is a major pro-inflammatory and immunoregulatory protein that stimulates fibroblast differentiation and proliferation, the production of prostaglandins, collagenase and phospholipase by synovial cells and chondrocytes, basophil and eosinophil degranulation and neutrophil activation. (Oppenheim, J. H. et al, Immunology Today, 7, pp. 45-56, 1986). As such, it is involved in the pathogenesis of chronic and acute inflammatory and autoimmune diseases. IL-1 is predominantly produced by peripheral blood monocytes as part of the inflammatory response and exists in two distinct agonist forms: IL-1 alpha (IL1A), IL-1 beta (IL1B) (Mosely, B. S. et al., Proc. Nat. Acad. Sci., 84, pp. 4572-4576 1987; Lonnemann, G. et al., Eur. J. Immunol., 19, pp. 1531-1536 1989).

IL1B is synthesized as a biologically inactive precursor, pIL1B. pIL1B lacks a conventional leader sequence and is not processed by a signal peptidase. (March, C. J., Nature, 315, pp. 641-647 1985). Instead, pIL1B is cleaved by caspase 1 to produce the biologically active form found in human serum and synovial fluid. (Sleath, P. R., et al., J. Biol. Chem., 265, pp. 14526-14528 1992; A. D. Howard et al., J. Immunol., 147, pp. 2964-2969 1991).

IL1B over-production has been implicated in the pathogenesis of a variety of diseases such as rheumatoid arthritis and osteoarthritis. IL1B has been shown to increase cell migration into the inflamed synovium of joints by the up-regulation of adhesion molecules, the stimulation of the production of prostaglandins and metalloproteinase, the inhibition of collagen and proteoglycan synthesis, and the stimulation of osteoclastic bone resorption. Because of these properties, IL-1 is one of the primary mediators of bone and cartilage destruction in arthritis.

### SUMMARY

Due to IL1B's function as a broad pro-inflammatory cytokine, IL1B blockade may potentially provide therapeutic benefit in atopic dermatitis, Epidermolysis bullosa, uveitis, Gout, Polymyalgia rheumatica, Osteoarthritis, Systemic-onset juvenile idiopathic arthritis, Schnitzler syndrome, Familial Mediterranean fever, Cryopyrin-associated periodic syndrome (CAPS), Hyper-IgD syndrome (HIDS), TNF receptor-associated periodic syndrome (TRAPS), Type 2 diabetes, Proliferative diabetic retinopathy, Wet age-related macular degeneration, Chronic obstructive pulmonary disease, Type 1 diabetes, Urticarial vasculitis, Pyoderma gangrenosum, Dry eye syndrome, Acne vulgaris, as well as improve arterial function in atherosclerosis in type 2 diabetes and reduce cardiovascular risk in type 2 diabetes.

An antisense based therapy targeting human IL-1B in keratinocytes may prove beneficial for diseases exhibiting excessive inflammation, including those listed above. In an effort to find an effective Spherical Nucleic Acid (SNA) construct, in vitro primary human neonatal foreskin keratinocytes (HFKs) were treated with antisense oligonucleotide SNA compounds targeted against human IL1B mRNA (GeneBank accession number NM_000576, SEQ ID NO: 1).

In some aspects the invention is a single-stranded modified oligonucleotide having or consisting of 10-30 linked nucleosides and having: a gap segment consisting of two to eight linked deoxynucleosides; a 5′ wing segment consisting of linked nucleosides; and a 3′ wing segment consisting of linked nucleosides; wherein the gap segment is positioned between the 5′ wing segment and the 3′ wing segment; wherein at least some nucleosides of each wing segment comprises a modified nucleotide; wherein at least 5 of the internucleoside linkages within the gap segment and the linkages connecting the gap segment to the 3′ wing segments are phosphorothioate linkages (*); and the internucleoside linkages connecting the rest of the nucleosides of both the 5′ and 3′ wing segments are phosphodiester linkages; and wherein the nucleobase sequence of the oligonucleotide consists of 10-30 contiguous nucleobases complementary to an equal length portion of SEQ ID NO: 1, or a pharmaceutically acceptable salt thereof.

In some embodiments the nucleobase sequence of the oligonucleotide is mGmUmAmGmUmGG*T*G*G*T*/iMe-dC/*mGmGmAmGmAmU (SEQ ID NO: 43), mAmGmAmUmCmCT*C*T*T*A*G*mCmAmCmUmAmC (SEQ ID NO: 3), mGmAmAmGmGmAG*C*A*C*T*T*mCmAmUmCmUmG (SEQ ID NO: 6), mCmCmAmAmGmGC*C*A*C*A*G*mGmUmAmUmUmU (SEQ ID NO: 7), or mUmCmCmAmGmCT*T*G*T*T*A*mUmUmGmAmUmU (SEQ ID NO: 10).

In some embodiments the oligonucleotide is 18 nucleotides in length.

In other embodiments the oligonucleotide includes a molecular species at one of the ends. In some embodiments the molecular species is selected from the group consisting of a spacer, a lipid, a sterol, cholesterol, stearyl, C16 alkyl chain, bile acids, cholic acid, taurocholic acid, deoxycholate, oleyl litocholic acid, oleoyl cholenic acid, glycolipids, phospholipids, sphingolipids, isoprenoids, such as steroids, vitamins, such as vitamin E, saturated fatty acids, unsaturated fatty acids, fatty acid esters, such as triglycerides, pyrenes, porphyrines, Texaphyrine, adamantane, acridines, biotin, coumarin, fluorescein, rhodamine, Texas-Red, digoxygenin, dimethoxytrityl, t-butyldimethylsilyl, t-butyldiphenylsilyl, cyanine dyes (e.g. Cy3 or Cy5), Hoechst 33258 dye, psoralen, and ibuprofen. The molecular species may be connected directly to the compound through a linkage selected from the group consisting of phosphodiester, phosphorothioate, methylphosphonate, and amide linkages. Alternatively the molecular species may be connected indirectly to the compound through a linker.

In some embodiments the linker is a non-nucleotidic linker selected from the group consisting of abasic residues (dSpacer), oligoethyleneglycol, such as triethyleneglycol (spacer 9) or hexaethyleneglycol (spacer 18), and alkane-diol, such as butanediol.

In other embodiments the 3′ end of the oligonucleotide is connected to 2 consecutive linkers that are hexaethyleneglycol (spacer 18), the first hexaethyleneglycol connected to the 3′ end of the oligonucleotide, the second hexaethyleneglycol connected to the first hexaethyleneglycol and the second hexaethyleneglycol is connected to a cholesterol.

The gap segment in some embodiments has six linked deoxynucleosides. In other embodiments all of the nucleosides of each wing segment comprise a modified nucleotide. In yet other embodiments each nucleoside of each wing segment comprises a 2′O-methyl ribonucleoside (m).

In some aspects the invention is an oligonucleotide comprising mGmUmAmGmUmGG*T*G*G*T*/iMe-dC/*mGmGmAmGmAmU/isp18//isp18//3CholTEG/(SEQ ID NO: 43), wherein m is a 2′O methyl, and wherein * is a phosphorothioate modification.

In some aspects the invention is an oligonucleotide comprising mAmGmAmUmCmCT*C*T*T*A*G*mCmAmCmUmAmC/isp18//isp18//3CholTEG/(SEQ ID NO: 3), wherein m is a 2′O methyl, and wherein * is a phosphorothioate modification.

In some aspects the invention is an oligonucleotide comprising mGmAmAmGmGmAG*C*A*C*T*T*mCmAmUmCmUmG/isp18//isp18//3CholTEG/(SEQ ID NO: 6), wherein m is a 2′O methyl, and wherein * is a phosphorothioate modification.

In some aspects the invention is an oligonucleotide comprising mCmCmAmAmGmGC*C*A*C*A*G*mGmUmAmUmUmU/isp18//isp18//3CholTEG/(SEQ ID NO: 7), wherein m is a 2′O methyl, and wherein * is a phosphorothioate modification.

In other aspects the invention is an oligonucleotide comprising mUmCmCmAmGmCT*T*G*T*T*A*mUmUmGmAmUmU/isp18//isp18//3CholTEG/(SEQ ID NO: 10), wherein m is a 2′O methyl, and wherein * is a phosphorothioate modification.

In yet other aspects, the invention is a stable self-assembling nanostructure, comprising a liposomal core comprised of a lipid bilayer and having an oligonucleotide shell comprised of an antisense oligonucleotide 18 to 21 linked nucleosides in length targeted to IL-1 beta (IL1B) and linked on the oligonucleotide 3′ end to an isp18//isp18//3CholTEG, wherein the 3CholTEG interacts with lipids in the lipid bilayer such that the oligonucleotide is radially oriented and the 5′ end of the oligonucleotide faces externally from the nanostructure. In some embodiments the antisense oligonucleotide is 18 nucleotides in length. In other embodiments IL1B has a sequence of SEQ ID NO: 1.

In other embodiments the antisense oligonucleotide has 2′O methyl modifications.

In some embodiments the antisense oligonucleotide is selected from the group consisting of mGmUmAmGmUmGG*T*G*G*T*/iMe-dC/*mGmGmAmGmAmU (SEQ ID NO: 43), mAmGmAmUmCmCT*C*T*T*A*G*mCmAmCmUmAmC (SEQ ID NO: 3), mGmAmAmGmGmAG*C*A*C*T*T*mCmAmUmCmUmG (SEQ ID NO: 6), mCmCmAmAmGmGC*C*A*C*A*G*mGmUmAmUmUmU (SEQ ID NO: 7), and mUmCmCmAmGmCT*T*G*T*T*A*mUmUmGmAmUmU (SEQ ID NO: 10) wherein—refers to a phosphodiester bond, * refers to a phosphorothioate bond, and m refers to a 0 methyl.

A method for treating a disorder is provided in other aspects of the invention. The method involves administering to a subject having a disorder a stable self-assembling nanostructure, comprising a liposomal core comprised of a lipid bilayer and having an oligonucleotide shell comprised of an antisense oligonucleotide 18 to 21 linked nucleosides in length targeted to IL-1 beta (IL1B) and linked on the oligonucleotide 3′ end to an isp18//isp18//3CholTEG, wherein the 3CholTEG interacts with lipids in the lipid bilayer such that the oligonucleotide is radially oriented and the 5′ end of the oligonucleotide faces externally from the nanostructure in an effective amount to treat the disorder.

In some embodiments the disorder is an inflammatory disorder. In other embodiments the disorder is selected from atopic dermatitis, Epidermolysis bullosa, uveitis, Gout, Polymyalgia rheumatica, Osteoarthritis, Systemic-onset juvenile idiopathic arthritis, Schnitzler syndrome, Familial Mediterranean fever, Cryopyrin-associated periodic syndrome (CAPS), Hyper-IgD syndrome (HIDS), TNF receptor-associated periodic syndrome (TRAPS), Type 2 diabetes, Proliferative diabetic retinopathy, Wet age-related macular degeneration, Chronic obstructive pulmonary disease, Type 1 diabetes, Urticarial vasculitis, Pyoderma gangrenosum, Dry eye syndrome, Acne vulgaris, as well as improve arterial function in atherosclerosis in type 2 diabetes and reduce cardiovascular risk in type 2 diabetes.

A method for reducing expression levels of IL1B in vivo, is also provided. The method involves administering to a subject a pharmaceutical composition comprising a stable self-assembling nanostructure, comprising a liposomal core comprised of a lipid bilayer and having an oligonucleotide shell comprised of an antisense oligonucleotide 18 to 21 linked nucleosides in length targeted to IL-1 beta (IL1B) and linked on the oligonucleotide 3′ end to an isp18//isp18//3CholTEG, wherein the 3CholTEG interacts with lipids in the lipid bilayer such that the oligonucleotide is radially oriented and the 5′ end of the oligonucleotide faces externally from the nanostructure in an effective amount to reduce IL1B levels in vivo. In some embodiments the subject is a mammal or human.

In other aspects the invention is method for reducing expression levels of IL1B in vitro, comprising: contacting a cell with a stable self-assembling nanostructure, comprising a liposomal core comprised of a lipid bilayer and having an oligonucleotide shell comprised of an antisense oligonucleotide 18 to 21 linked nucleosides in length targeted to IL-1 beta (IL1B) and linked on the oligonucleotide 3′ end to an isp18//isp18//3CholTEG, wherein the 3CholTEG interacts with lipids in the lipid bilayer such that the oligonucleotide is radially oriented and the 5′ end of the oligonucleotide faces externally from the nanostructure in an effective amount to reduce IL1B levels in vitro. In some embodiments the cell is a keratinocyte.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 shows inhibition of IL1B mRNA expression. IL1B mRNA expression in human primary keratinocytes after 24 hours treatment, as measured by qRT-PCR (percent of CTL SNA; error bars omitted for visual clarity). Average of n=2 independent experiments, each consisting of triplicate treatment wells and technical duplicates in qRT-PCR. The oligonucleotides in the tested SNAs are hIL1b_178 (SEQ ID NO: 6), hILb_241-12m5C (SEQ ID NO: 43), hIL1b_599 (SEQ ID NO: 7), hIL1b_750 (SEQ ID NO: 10), and hIL1b_1040 (SEQ ID NO: 3). % GE=% gene expression; CTL=control.

FIG. 2 shows onset of IL1B mRNA knockdown. IL1B mRNA expression in human primary keratinocytes at the indicated time after initiating SNA treatment. Values are normalized to the AntiControl SNA at each time point. Mean±SEM of n=3 triplicate wells and technical duplicates on PCR. % GE=% gene expression; CTL=control.

FIG. 3 shows persistence of IL1B mRNA knockdown. IL1B mRNA expression in human primary keratinocytes at the indicated time after initiating SNA (hIL1b_178, SEQ ID NO: 6) treatment. Values are normalized to cells treated with the vehicle (PBS) at each time point. Mean±SEM of n=3 independent experiments, each of which consisted of triplicate wells and technical duplicates on PCR. CTL=control.

FIG. 4 shows the dose response curve of SNA compounds. Dose-response and IC₅₀ of IL-1β mRNA reduction in HFKs treated with anti-IL1B SNAs. Mean±SEM of n=4 (SNA1) or n=2 (SNA2) independent experiments. SNA1=hIL1B_178 (SEQ ID NO: 6), SNA2=hIL1B_241-12m5C (SEQ ID NO: 43). CTL=control.

FIGS. 5A-D show IL-1β knockdown in epidermolysis bullosa patient-derived cells. Dermal fibroblasts were obtained from a healthy donor and from two patients with dystrophic epidermolysis bullosa (EB), a disorder exhibiting dysregulated inflammation. Fibroblasts were stimulated with TNF protein to simulate the hyper-inflamed disease condition. After 24 hours of SNA treatment, IL-1β mRNA was measured by qRT-PCR. Results from one anti-IL-1β SNA in fibroblasts from two EB patients in a single experiment is shown, representative of the results seen in n=3 independent experiments using fibroblasts from two EB patients and two anti-IL-1β SNAs. Mean±SEM of triplicate wells are displayed. SNA2=hIL1B_241-12m5C. FIG. 5A shows baseline and TNF-stimulated IL-1β mRNA levels in healthy donor and EB fibroblasts. FIG. 5B shows IL-1β mRNA knockdown in healthy donor fibroblasts. FIG. 5C shows IL-1β mRNA knockdown in EB patient-derived fibroblasts, normalized to healthy donor. FIG. 5D shows IL-1β mRNA knockdown in EB patient-derived fibroblasts, normalized to CTL SNA. CTL=control.

### DETAILED DESCRIPTION

The invention in some aspects relates to antisense inhibitors of IL1B. IL1B plays an important role in inflammation. Antisense technology is a useful means for reducing the expression of specific gene products by targeting a messenger RNA (mRNA) and preventing translation of the mRNA. However, the selection of specific therapeutically functional antisense oligonucleotides (ASOs) can be challenging. Further, ASO that are therapeutically active in a linear form do not necessarily retain activity when formulated as a nanoparticle or other type of three dimensional presentation format.

It has been discovered in aspects of the invention that Spherical Nucleic Acids (SNAs) surface functionalized with antisense oligonucleotides (ASOs) having appropriate structural properties can mediate highly effective gene knockdown of the intended target mRNA. For example, it is demonstrated herein that IL1B expression can be inhibited through the use of SNAs targeting human IL1B (anti-IL1B-SNAs) mRNA in keratinocytes with no associated toxicity or immune-stimulatory effects. In some embodiments, the anti-IL1B SNAs inhibit IL1B mRNA expression in a dose-dependent manner. As described herein, the oligonucleotides arranged in an SNA geometry exhibit enhanced penetration and increased cellular uptake.

An “IL1B inhibitor” as used herein refers to a nucleic acid based agent which interferes with IL1B activity. In particular, the IL1B antisense inhibitors or IL1B antisense oligonucleotides of the invention reduce the expression of the IL1B gene.

The IL1B inhibitors of the invention are antisense nucleic acids. Antisense nucleic acids typically include modified or unmodified RNA, DNA, or mixed polymer nucleic acids, and primarily function by specifically binding to matching sequences resulting in modulation of peptide synthesis. Antisense nucleic acids bind to target RNA by Watson Crick base-pairing and block gene expression by preventing ribosomal translation of the bound sequences either by steric blocking or by activating RNase H enzyme. Antisense molecules may also alter protein synthesis by interfering with RNA processing or transport from the nucleus into the cytoplasm.

As used herein, the term “antisense nucleic acid” or “antisense oligonucleotide” describes a nucleic acid that hybridizes under physiological conditions to DNA comprising a particular gene or to an mRNA transcript of that gene in this case IL1B and, thereby, inhibits the transcription of that gene and/or the translation of that mRNA. The antisense molecules are designed so as to interfere with transcription or translation of a target gene upon hybridization with the target gene or transcript. Those skilled in the art will recognize that the exact length of the antisense oligonucleotide and its degree of complementarity with its target will depend upon the specific target selected, including the sequence of the target and the particular bases which comprise that sequence.

“Inhibition of gene expression” refers to the absence or observable decrease in the level of protein and/or mRNA product from a target gene, such as the IL1B gene. “Specificity” refers to the ability to inhibit the target gene without manifest effects on other genes of the cell. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS).

The antisense oligonucleotides of the invention inhibit IL1B expression. Depending on the assay, quantitation of the amount of gene expression allows one to determine a degree of inhibition which is greater than 5%, 10%, 15%, 20%, 25%, 30%, 35%, 40%, 45%, 50%, 55%, 60%, 65%, 70%, 75%, 80%, 85%, 90%, 95%, or 100% as compared to a cell not treated according to the present invention. As an example, the efficiency of inhibition may be determined by assessing the amount of gene product in the cell.

The ASOs described herein include bioequivalent compounds, salts and prodrugs thereof. The term bioequivalent compounds, including pharmaceutically acceptable salts and prodrugs as used herein refers to antisense oligonucleotides having the same primary structure as the antisense oligonucleotide of interest, but including salt forms or structures which can be cleaved or modified to have the same type of biological effect as the antisense oligonucleotide of interest. This is intended to encompass any pharmaceutically acceptable salts, esters, or salts of such esters, or any other compound which, upon administration to an animal including a human, is capable of providing (directly or indirectly) the biologically active metabolite or residue thereof.

“Pharmaceutically acceptable salts” are physiologically and pharmaceutically acceptable salts of the nucleic acids of the invention: i.e., salts that retain the desired biological activity of the compound of interest and do not impart undesired toxicological effects thereto. Pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

The compounds of the invention may also be prepared to be delivered in a “prodrug” form. A “prodrug” is a therapeutic agent that is prepared in an inactive form that is converted to an active form (i.e., drug) within the body or cells thereof by the action of endogenous enzymes or other chemicals and/or conditions.

The antisense oligonucleotides of the invention are IL1B antisense oligonucleotides. An antisense IL1B oligonucleotide refers to a compound having a sequence of nucleotide bases and a subunit-to-subunit backbone that allows the antisense oligonucleotide to hybridize to a IL1B target mRNA sequence typically by Watson-Crick base pairing, to form an RNA:oligomer heteroduplex within the target sequence.

The specific hybridization of an antisense oligonucleotide with its target nucleic acid, IL1B mRNA, interferes with the normal function of the IL1B mRNA. This modulation of function of a target nucleic acid by compounds which specifically hybridize to it is generally referred to as “antisense”. The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of IL1B protein. In the context of the present invention, “modulation” means a decrease or inhibition in the expression of a gene.

An antisense oligonucleotide “specifically hybridizes” to a target polynucleotide if the oligonucleotide hybridizes to the IL1B target under physiological conditions, with a thermal melting temperature (Tm) substantially greater than 37° C., preferably at least 45° C., and typically 50° C.-80° C. or higher. Such hybridization preferably corresponds to stringent hybridization conditions, selected to be about 10° C., and preferably about 50° C. lower than the Tm for the specific sequence at a defined ionic strength and pH. At a given ionic strength and pH, the Tm is the temperature at which 50% of a target sequence hybridizes to a complementary polynucleotide.

Polynucleotides are described as “complementary” to one another when hybridization occurs in an antiparallel configuration between two single-stranded polynucleotides. A double-stranded polynucleotide can be “complementary” to another polynucleotide, if hybridization can occur between one of the strands of the first polynucleotide and the second. Complementarity (the degree that one polynucleotide is complementary with another) is quantifiable in terms of the proportion of bases in opposing strands that are expected to form hydrogen bonds with each other, according to generally accepted base-pairing rules. An antisense compound may be complementary to a target region of a target transcript even if the two bases sequences are not 100% complementary, as long as the heteroduplex structure formed between the compound and transcript has the desired Tm stability.

Identifying an antisense oligonucleotide that targets a particular nucleic acid may be a multistep process. The process usually begins with the identification of a nucleic acid sequence whose function is to be modulated. This may be, for example, a cellular gene (or mRNA transcribed from the gene) whose expression is associated with a particular inflammatory disorder or disease state. The targeting process also includes determination of a site or sites within this IL1B gene for the antisense interaction to occur such that the desired effect, e.g., detection or modulation of expression of the protein, will result.

In some embodiments, the inflammatory disorder treated in the present disclosure is selected from the group consisting of an autoimmune disease, an infectious disease, transplant rejection or graft-versus-host disease, malignancy, a pulmonary disorder, an intestinal disorder, a cardiac disorder, sepsis, a spondyloarthropathy, a metabolic disorder, anemia, pain, a hepatic disorder, a skin disorder, a nail disorder, rheumatoid arthritis, psoriasis, psoriasis in combination with psoriatic arthritis, ulcerative colitis, Crohn's disease, vasculitis, Behcet's disease, ankylosing spondylitis, asthma, chronic obstructive pulmonary disorder (COPD), idiopathic pulmonary fibrosis (IPF), restenosis, diabetes, anemia, pain, a Crohn's disease-related disorder, juvenile rheumatoid arthritis (JRA), a hepatitis C virus infection, psoriatic arthritis, and chronic plaque psoriasis. Non-limiting examples of autoimmune disorders include the autoimmune disorder is selected from the group consisting of rheumatoid arthritis, rheumatoid spondylitis, osteoarthritis, gouty arthritis, allergy, multiple sclerosis, autoimmune diabetes, autoimmune uveitis, and nephritic syndrome.

In some embodiments, antisense oligonucleotides are designed to target human Interleukin 1B (IL1B), for instance, the nucleotide sequence of SEQ ID NO: 1.

The nanostructures descried herein may be stable self-assembling nanostructures. For instance the nanostructure may be an antisense oligonucleotide of 18-21 nucleotides in length having a sequence described herein, wherein a hydrophobic group at the 3′ or 5′ terminus self-associates to form the core of the nanostructure in water or other suitable solvents. A hydrophobic group as used herein may include cholesterol, a cholesteryl or modified cholesteryl residue, adamantine, dihydrotesterone, long chain alkyl, long chain alkenyl, long chain alkynyl, olely-lithocholic, cholenic, oleoyl-cholenic, palmityl, heptadecyl, myrisityl, bile acids, cholic acid or taurocholic acid, deoxycholate, oleyl litocholic acid, oleoyl cholenic acid, glycolipids, phospholipids, sphingolipids, isoprenoids, such as steroids, vitamins, such as vitamin E, fatty acids either saturated or unsaturated, fatty acid esters, such as triglycerides, pyrenes, porphyrines, Texaphyrine, adamantane, acridines, biotin, coumarin, fluorescein, rhodamine, Texas-Red, digoxygenin, dimethoxytrityl, t-butyldimethylsilyl, t-butyldiphenylsilyl, cyanine dyes (e.g. Cy3 or Cy5), Hoechst 33258 dye, psoralen, or ibuprofen.

The antisense oligonucleotides typically have a length of 10-30 or 15-20 bases, which is generally long enough to have one complementary sequence in the mammalian genome. Additionally, antisense compounds having a length of at least 12, typically at least 15 nucleotides in length hybridize well with their target mRNA. Thus, the antisense oligonucleotides of the invention are typically in a size range of 8-100 nucleotides, more preferably 12-50 nucleotides in length. In some embodiments the antisense oligonucleotide are 10, 12, 14, 16, 18, 20, 22, 24, 26, 28, 30, 32, 34, 36, 38, 40, 42, 44, 46, 48, 50, 52, 54, 56, 58, or 60 nucleotides in length. In some embodiments the antisense oligonucleotides are in a size range of about 10-100, 10-80, 10-50, 10-40, 10-30, 10-25, 10-20, 12-100, 12-80, 12-50, 12-40, 12-30, 12-28, 12-25, 12-20, 14-100, 14-80, 14-50, 14-30, 14-28, 14-25, 14-20, 16-50, 16-40, 16-30, 16-28, 16-25, 16-20, 18-50, 18-40, 18-30, 18-28, 18-26, 18-25, 18-24, 18-20, 20-100, 20-90, 20-80, 20-70, 20-60, 20-50, 20-40, 20-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 nucleotides in length. In some embodiments of the invention the antisense oligonucleotides are of 18-19 nucleotides in length. The antisense oligonucleotides may include further nucleotides on the 5′ and/or 3′ end of the oligonucleotide. However an antisense oligonucleotide that is limited to 18 nucleotides in length, for example, does not have any additional nucleotides on the 5′ or 3′ end of the molecule. Other non-nucleotide molecules may be linked covalently or non-covalently to the 5′ and/or 3′ end of those oligonucleotides.

The terms “nucleic acid” and “oligonucleotide” are used interchangeably to mean multiple nucleotides (i.e., molecules comprising a sugar (e.g., ribose or deoxyribose) linked to a phosphate group and to an exchangeable organic base, which is either a substituted pyrimidine (e.g., cytosine (C), thymine (T) or uracil (U)) or a substituted purine (e.g., adenine (A) or guanine (G)). As used herein, the terms “nucleic acid” and “oligonucleotide” refer to oligoribonucleotides as well as oligodeoxyribonucleotides. The terms “nucleic acid” and “oligonucleotide” shall also include polynucleosides (i.e., a polynucleotide minus the phosphate) and any other organic base containing polymer. Nucleic acid molecules are preferably synthetic (e.g., produced by nucleic acid synthesis). The oligonucleotides may be any size useful for producing antisense effects. In some embodiments they are 18-23 nucleotides in length. In other embodiments the antisense oligonucleotide is 18 nucleotides in length.

The terms “nucleic acid” and “oligonucleotide” may also encompass nucleic acids or oligonucleotides with substitutions or modifications, such as in the bases and/or sugars. For example, they include nucleic acids having backbone sugars that are covalently attached to low molecular weight organic groups other than a hydroxyl group at the 2′ position and other than a phosphate group or hydroxy group at the 5′ position. Thus modified nucleic acids may include a 2′-O-alkylated ribose group. In addition, modified nucleic acids may include sugars such as hexose, 2′-F hexose, 2′-amino ribose, CEt-LNA, arabinose or 2′-fluoroarabinose instead of ribose. Thus the nucleic acids may be heterogeneous in backbone composition thereby containing any possible combination of polymer units linked together such as peptide-nucleic acids (which have an amino acid backbone with nucleic acid bases). Other examples are described in more detail below.

The oligonucleotides may be DNA, RNA, PNA, LNA, ENA or hybrids including any chemical or natural modification thereof. Chemical and natural modifications are well known in the art. Such modifications include, for example, modifications designed to increase binding to a target strand (i.e., increase their melting temperatures), to assist in identification of the oligonucleotide or an oligonucleotide-target complex, to increase cell penetration, to stabilize against nucleases and other enzymes that degrade or interfere with the structure or activity of the oligonucleotides, to provide a mode of disruption (a terminating event) once sequence-specifically bound to a target, and to improve the pharmacokinetic properties of the oligonucleotide.

Modifications include, but are not limited to, for example, (a) end modifications, e.g., 5′ end modifications (phosphorylation dephosphorylation, conjugation, inverted linkages, etc.), 3′ end modifications (conjugation, DNA nucleotides, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, as well as (d) internucleoside linkage modifications, including modification or replacement of the phosphodiester linkages. To the extent that such modifications interfere with translation (i.e., results in a reduction of 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more in translation relative to the lack of the modification—e.g., in an in vitro translation assay), the modification may not be optimal for the methods and compositions described herein.

Non-limiting examples of modified internucleoside linkages include phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

In some embodiments, the modified oligonucleotide is a single-stranded modified oligonucleotide. In some embodiments, the single-stranded modified oligonucleotide consists of 10-30, 10-35, 10-40, 10-45, 10-50, 10-60, 10-70, 10-80, 10-90, 10-100, 20-30, 20-35, 20-40, 20-45, 20-50, 20-60, 20-70, 20-80, 20-90, 20-100, 30-35, 30-40, 30-45, 30-50, 30-60, 30-70, 30-80, 30-90, 30-100, 40-45, 40-50, 40-60, 40-70, 40-80, 40-90, 40-100, 50-60, 50-65, 50-70, 50-75, 50-80, 50-85, 50-90, 50-95, 50-100, 60-65, 60-70, 60-75, 60-80, 60-85, 60-90, 60-95, 60-100, 70-75, 70-80, 70-85, 70-90, 70-95, 70-100, 80-85, 80-90, 80-95, 80-100, 85-90, 85-95, 85-100, 90-95, 90-100, 95-100 or more than 100 linked nucleosides and has a gap segment. In some embodiments, a gap segment refers to one or more linked nucleic acids consisting of deoxynucleosides located at the center or near the center of a modified oligonucleotide, such as a single-stranded modified oligonucleotide. In some embodiments, the gap segment consists of 2-3, 2-4, 2-5, 2-6, 2-7, 2-8, 2-9, 2-10, 2-20, 2-30, 2-40, 2-50, 5-10, 5-50, 5-30, 5-40, 5-50, 10-20, 10-30, 10-40 or 10-50, 20-50, 30-50, 40-50 or 45-50 linked deoxynucleosides.

A 5′ wing segment corresponds to the linked nucleic acids (e.g., nucleosides) from the 5′-end of a modified oligonucleotide to the nucleic acid before the first nucleic acid at the 5′-end of the gap segment. A 3′ wing segment corresponds to the linked nucleic acids (e.g., nucleosides) after the last nucleic acid at the 3′ end of the gap segment to the last nucleic acid at the 3′ end of the modified oligonucleotide.

The gap segment is positioned between the 5′ wing segment and the 3′ wing segment. In some embodiments, at least one nucleoside of the 5′ wing segment and/or at least one nucleoside of the 3′ wing segment comprises a modified nucleoside. In some embodiments, the internucleoside linkages within the gap segment and the linkages connecting the gap segment to the 3′ wing segment and/or the 5′ wing segment are all phosphorothioate linkages (*). In some embodiments, the internucleoside linkages connecting the rest of the nucleosides of both the 5′ and 3′ wing segments are phosphodiester linkages. In some embodiments, the nucleosides in the modified oligonucleotide are modified with a 2′ O-methyl group. The nucleosides in the modified oligonucleoside can also be modified with any other modification described herein.

In some embodiments, the nucleobase sequence of the modified oligonucleotide consists of 10-30, 10-35, 10-40, 10-45, 10-50, 10-60, 10-70, 10-80, 10-90, 10-100, 20-30, 20-35, 20-40, 20-45, 20-50, 20-60, 20-70, 20-80, 20-90, 20-100, 30-35, 30-40, 30-45, 30-50, 30-60, 30-70, 30-80, 30-90, 30-100, 40-45, 40-50, 40-60, 40-70, 40-80, 40-90, 40-100, 50-60, 50-65, 50-70, 50-75, 50-80, 50-85, 50-90, 50-95, 50-100, 60-65, 60-70, 60-75, 60-80, 60-85, 60-90, 60-95, 60-100, 70-75, 70-80, 70-85, 70-90, 70-95, 70-100, 80-85, 80-90, 80-95, 80-100, 85-90, 85-95, 85-100, 90-95, 90-100, 95-100 or more than 100 linked nucleosides and has a gap segment complementary to an equal length portion of the coding sequence (e.g., cDNA) of the IL1B (e.g., cDNA sequence of IL1B is represented by SEQ ID NO: 1 or a pharmaceutically acceptable salt thereof.

Modified internucleoside linkages that do not include a phosphorus atom therein have internucleoside linkages that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Substituted sugar moieties include, but are not limited to one of the following at the 2′ position: H (deoxyribose); OH (ribose); F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl.

A chemically or naturally modified oligonucleotide may include, for example, at least one nucleotide modified at the 2′ position of the sugar, most preferably a 2′-O-alkyl, 2′-O-alkyl-O-alkyl or 2′-fluoro-modified nucleotide or an end cap. In other embodiments, RNA modifications include 2′-fluoro, 2′-amino and 2′ O-methyl modifications on the ribose of pyrimidines, abasic residues or an inverted base at the 3′ end of the RNA.

The oligonucleotides useful according to the invention may include a single modified nucleoside. In other embodiments the oligonucleotide may include at least two modified nucleosides, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 or more nucleosides, up to the entire length of the oligonucleotide.

Nucleosides or nucleobases include the natural purine bases adenine (A) and guanine (G), and the pyrimidine bases thymine (T), cytosine (C) and uracil (U). Modified nucleosides include other synthetic and natural nucleobases such as inosine, xanthine, hypoxanthine, nubularine, isoguanisine, tubercidine, 2-(halo)adenine, 2-(alkyl)adenine, 2-(propyl)adenine, 2 (amino)adenine, 2-(aminoalkyll)adenine, 2 (aminopropyl)adenine, 2 (methylthio) N6 (isopentenyl)adenine, 6 (alkyl)adenine, 6 (methyl)adenine, 7 (deaza)adenine, 8 (alkenyl)adenine, 8-(alkyl)adenine, 8 (alkynyl)adenine, 8 (amino)adenine, 8-(halo)adenine, 8-(hydroxyl)adenine, 8 (thioalkyl) adenine, 8-(thiol)adenine, N6-(isopentyl)adenine, N6 (methyl)adenine, N6, N6 (dimethyl)adenine, 2-(alkyl)guanine, 2 (propyl)guanine, 6-(alkyl)guanine, 6 (methyl)guanine, 7 (alkyl)guanine, 7 (methyl)guanine, 7 (deaza)guanine, 8 (alkyl)guanine, 8-(alkenyl)guanine, 8 (alkynyl)guanine, 8-(amino)guanine, 8 (halo)guanine, 8-(hydroxyl)guanine, 8 (thioalkyl)guanine, 8-(thiol)guanine, N (methyl)guanine, 2-(thio)cytosine, 3 (deaza) 5 (aza)cytosine, 3-(alkyl)cytosine, 3 (methyl)cytosine, 5-(alkyl)cytosine, 5-(alkynyl)cytosine, 5 (halo)cytosine, 5 (methyl)cytosine, 5 (propynyl)cytosine, 5 (propynyl)cytosine, 5 (trifluoromethyl)cytosine, 6-(azo)cytosine, N4 (acetyl)cytosine, 3 (3 amino-3 carboxypropyl)uracil, 2-(thio)uracil, 5 (methyl) 2 (thio)uracil, 5 (methylaminomethyl)-2 (thio)uracil, 4-(thio)uracil, 5 (methyl) 4 (thio)uracil, 5 (methylaminomethyl)-4 (thio)uracil, 5 (methyl) 2,4 (dithio)uracil, 5 (methylaminomethyl)-2,4 (dithio)uracil, 5 (2-aminopropyl)uracil, 5-(alkyl)uracil, 5-(alkynyl)uracil, 5-(allylamino)uracil, 5 (aminoallyl)uracil, 5 (aminoalkyl)uracil, 5 (guanidiniumalkyl)uracil, 5 (1,3-diazole-1-alkyl)uracil, 5-(cyanoalkyl)uracil, 5-(dialkylaminoalkyl)uracil, 5 (dimethylaminoalkyl)uracil, 5-(halo)uracil, 5-(methoxy)uracil, uracil-5 oxyacetic acid, 5 (methoxycarbonylmethyl)-2-(thio)uracil, 5 (methoxycarbonyl-methyl)uracil, 5 (propynyl)uracil, 5 (propynyl)uracil, 5 (trifluoromethyl)uracil, 6 (azo)uracil, dihydrouracil, N3 (methyl)uracil, 5-uracil (i.e., pseudouracil), 2 (thio)pseudouracil, 4 (thio)pseudouracil, 2,4-(dithio)psuedouracil, 5-(alkyl)pseudouracil, 5-(methyl)pseudouracil, 5-(alkyl)-2-(thio)pseudouracil, 5-(methyl)-2-(thio)pseudouracil, 5-(alkyl)-4 (thio)pseudouracil, 5-(methyl)-4 (thio)pseudouracil, 5-(alkyl)-2,4 (dithio)pseudouracil, 5-(methyl)-2,4 (dithio)pseudouracil, 1 substituted pseudouracil, 1 substituted 2(thio)-pseudouracil, 1 substituted 4 (thio)pseudouracil, 1 substituted 2,4-(dithio)pseudouracil, 1 (aminocarbonylethylenyl)-pseudouracil, 1 (aminocarbonylethylenyl)-2(thio)-pseudouracil, 1 (aminocarbonylethylenyl)-4 (thio)pseudouracil, 1 aminocarbonylethylenyl)-2,4-(dithio)pseudouracil, 1 (arninoalkylarninocarbonylethylenyl)-pseudouracil, 1 (arninoalkylarnino-carbonylethylenyl)-2(thio)-pseudouracil, 1(arninoalkylarninocarbonylethylenyl)-4 (thio)pseudouracil, 1 (arninoalkylarninocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(arninoalkylhydroxy)-1,3-(diaza)-2-(oxo)-phenoxazin-1-yl, 7-(arninoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(arninoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, diiluorotolyl, 4-(iluoro)-6-(methyl)benzimidazole, 4-(methyl)benzimidazole, 6-(azo)thymine, 2-pyridinone, 5 nitroindole, 3 nitropyrrole, 6-(aza)pyrimidine, 2 (amino)purine, 2,6-(diamino) purine, 5 substituted pyrimidines, N2-substituted purines, N6-substituted purines, 06-substituted purines, substituted 1,2,4-triazoles, pyrrolo-pyrimidin-2-on-3-yl, 6-phenyl-pyrrolo-pyrimidin-2-on-3-yl, para-substituted-6-phenyl-pyrrolo-pyrimidin-2-on-3-yl, ortho-substituted-6-phenyl-pyrrolo-pyrimidin-2-on-3-yl, bis-ortho-substituted-6-phenyl-pyrrolo-pyrimidin-2-on-3-yl, para-(aminoalkylhydroxy)-6-phenyl-pyrrolo-pyrimidin-2-on-3-yl, ortho-(aminoalkylhydroxy)-6-phenyl-pyrrolo-pyrimidin-2-on-3-yl, bis-ortho-(aminoalkylhydroxy)-6-phenyl-pyrrolo-pyrimidin-2-on-3-yl, pyridopyrimidin-3-yl, 2-oxo-7-amino-pyridopyrimidin-3-yl, 2-oxo-pyridopyrimidine-3-yl, or any O-alkylated or N-alkylated derivatives thereof.

The antisense oligonucleotides of the invention may be chimeric oligonucleotides. Chimeric antisense compounds of the invention may be formed as composite structures of two or more oligonucleotides, modified oligonucleotides, oligonucleotides and/or oligonucleotide mimetics as described above. Such compounds have also been referred to in the art as hybrids or mixed backbone or chimeric or gapmers. In particular a gapmer is an oligonucleotide that has at least three discrete portions, two of which are similar i.e. include one or more backbone modifications, and surround a region that is distinct, i.e., does not include backbone modifications.

The oligonucleotides may include a molecular species at one or both ends, i.e., at the 3′ and/or 5′ end. A molecular species as used herein refers to any compound that is not a naturally occurring or non-naturally occurring nucleotide. Molecular species include but are not limited to a spacer, a lipid, a sterol, lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, an octadecylamine or hexylamino-carbonyl-oxycholesterol moiety, stearyl, C16 alkyl chain, bile acids, cholic acid, taurocholic acid, deoxycholate, oleyl litocholic acid, oleoyl cholenic acid, glycolipids, phospholipids, sphingolipids, isoprenoids, such as steroids, vitamins, such as vitamin E, saturated fatty acids, unsaturated fatty acids, fatty acid esters, such as triglycerides, pyrenes, porphyrines, Texaphyrine, adamantane, acridines, biotin, coumarin, fluorescein, rhodamine, Texas-Red, digoxygenin, dimethoxytrityl, t-butyldimethylsilyl, t-butyldiphenylsilyl, cyanine dyes (e.g. Cy3 or Cy576), Hoechst 33258 dye, psoralen, or ibuprofen.

The molecular species may be attached at various positions of the oligonucleotide. As described above, the molecular species may be linked to the 2′-end, 3′-end or 5′-end of the oligonucleotide, where it also serves the purpose to enhance the stability of the oligomer against 3′- or 5′-exonucleases. Alternatively, it may be linked to an internal nucleotide or a nucleotide on a branch. The molecular species may be attached to a 2′-position of the nucleotide. The molecular species may also be linked to the heterocyclic base of the nucleotide.

The molecular species may be connected to the oligonucleotide by a linker moiety. Optionally the linker moiety is a non-nucleotidic linker moiety. Non-nucleotidic linkers are e.g. abasic residues (dSpacer), oligoethyleneglycol, such as triethyleneglycol or hexaethyleneglycol, or alkane-diol, such as butanediol. The spacer units are preferably linked by phosphodiester, phosphorodithioate or phosphorothioate bonds. The linker units may appear just once in the molecule or may be incorporated several times, e.g. via phosphodiester, phosphorothioate, methylphosphonate, or amide linkages.

The oligonucleotide of the invention (separate from the linkers connecting nucleotides to the molecular species) may also contain non-nucleotidic linkers, in particular abasic linkers (dSpacers), trietyhlene glycol units or hexaethylene glycol units. Further preferred linkers are alkylamino linkers, such as C3, C6, C12 aminolinkers, and also alkylthiol linkers, such as C3 or C6 thiol linkers.

Toxicity and efficacy of the prophylactic and/or therapeutic protocols of the present invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography. A number of studies have examined the optimal dosages for antisense oligonucleotides.

In certain embodiments, pharmaceutical compositions may comprise, for example, at least about 0.000001% (w/w) of an active compound. In other embodiments, the active compound may comprise between about 2% to about 75% of the weight of the unit (w/w), or between about 25% to about 60%, for example, and any range derivable therein. In some embodiments, the active compound (e.g., oligonucleotide or nanostructure) described herein comprises between 0.000001% and 0.00001%, between 0.00001% and 0.0001%, between 0.0001% and 0.001%, between 0.001% and 0.01%, between 0.01% and 0.1%, between 0.1% and 1%, between 1% and 5%, between 5% and 10%, between 10% and 15%, between 15% and 20%, between 20% and 25%, between 25% and 30%, between 30% and 40%, between 40% and 50% (w/w), and any range derivable in between. In some embodiments, the active compound (e.g., oligonucleotide or nanostructure) described herein comprises 0.00007%, 0.007%, 0.01%, 0.1%, 1% (w/w)

Subject doses of the compounds described herein typically range from about 0.1 μg to 10,000 mg, more typically from about 1 μg/day to 8000 mg, and most typically from about 10 μg to 100 μg. Stated in terms of subject body weight, typical dosages range from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above. The absolute amount will depend upon a variety of factors including the concurrent treatment, the number of doses and the individual patient parameters including age, physical condition, size and weight. These are factors well known to those of ordinary skill in the art and can be addressed with no more than routine experimentation. It is preferred generally that a maximum dose be used, that is, the highest safe dose according to sound medical judgment.

Multiple doses of the molecules of the invention are also contemplated. In some instances, when the molecules of the invention are administered with another therapeutic, for instance, an anti-inflammatory agent, a sub-therapeutic dosage of either the molecules or the other agent, or a sub-therapeutic dosage of both, is used in the treatment of a subject having, or at risk of developing an inflammatory disorder. When the two classes of drugs are used together, the other agent may be administered in a sub-therapeutic dose to produce a desirable therapeutic result. A “sub-therapeutic dose” as used herein refers to a dosage which is less than that dosage which would produce a therapeutic result in the subject if administered in the absence of the other agent. Thus, the sub-therapeutic dose of a therapeutic agent is one which would not produce the desired therapeutic result in the subject in the absence of the administration of the molecules of the invention. Therapeutic doses of agents useful for treating inflammatory disorders are well known in the field of medicine. These dosages have been extensively described in references such as Remington's Pharmaceutical Sciences; as well as many other medical references relied upon by the medical profession as guidance for the treatment of infectious disease, cancer, and autoimmune disease. Therapeutic dosages of oligonucleotides have also been described in the art.

Dosing regimens may be several times a day, daily, every other day, weekly, biweekly any of the times there between or less frequently. The term “biweekly dosing” as used herein, refers to the time course of administering a substance (e.g., an anti-IL1B nucleic acid) to a subject once every two weeks. The oligonucleotides may be administered every 7-20 days, every 11-17 days, or every 13-15 days, for example.

In some embodiments, a compound (e.g., oligonucleotide, nanostructure, etc.) described herein is administered for 4, 8, 12, 16, 20, 24, 28, 32, 36, 40, 44, 48, 72, 96, 120, 240, 480 hours, or any ranges in between, per dose in a dosing schedule. In alternative embodiments, the time intervals between administration of single doses of a compound (e.g., oligonucleotide, nanostructure, etc.) described herein are 4, 8, 12, 16, 20, 24, 28, 32, 36, 40, 44, 48, 72, 96, 120, 240, 480 hours, or any ranges in between.

The oligonucleotides are administered in effective amounts. The effective amount of a compound of the invention in the treatment of a disease described herein may vary depending upon the specific compound used, the mode of delivery of the compound, and whether it is used alone or in combination. The effective amount for any particular application can also vary depending on such factors as the disease being treated, the particular compound being administered, the size of the subject, or the severity of the disease or condition. One of ordinary skill in the art can empirically determine the effective amount of a particular molecule of the invention without necessitating undue experimentation. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject.

As used herein, the terms “treat,” “treatment,” “treating,” or “amelioration” refer to therapeutic treatments, wherein the object is to reverse, alleviate, ameliorate, inhibit, slow down or stop the progression or severity of a condition or disorder. Treatment is generally “effective” if one or more symptoms or clinical markers are reduced as determined using standard techniques or procedures available to one of ordinary skill in the art. Alternatively, treatment is “effective” if the progression of the condition or disorder disclosed herein is reduced or halted, as determined by one of ordinary skill in the art. That is, “treatment” includes not just the improvement of symptoms or markers, but can also include a cessation or at least slowing of progress or worsening of symptoms that would be expected in absence of treatment. The term “treatment” of a disease also includes providing relief from the symptoms or side-effects of the disease (including palliative treatment). In some embodiments, the terms “treat,” “treatment,” “treating,” or “amelioration” refer to the prophylactic treatment of a subject at risk of having or developing a disease.

In some embodiments, the subject is a mammal. In some embodiments, the mammal is a vertebrate animal including, but not limited to, a mouse, rat, dog, cat, horse, cow, pig, sheep, goat, turkey, chicken, monkey, fish (e.g., aquaculture species, salmon, etc.). In some embodiments, the subject is human.

In some embodiments, the cell is contacted with an oligonucleotide, a nanostructure, or a mASO-SNA described herein at a concentration of at least 0.001 nM, at least 0.01 nM, at least 0.1 nM, at least 1 nM, at least 10 nM, at least 100 nM, at least 1000 nM, at least 10 μM, at least 100 μM, at least 1000 μM, or above 1000 μM. In some embodiments, the cell is contacted with an oligonucleotide, a nanostructure, or a mASO-SNA described herein at a concentration range of 0.001 nM to 0.01 nM, 0.01 nM to 0.1 nM, 0.1 nM to 1 nM, 1 nM to 10 nM, 10 nM to 100 nM, 100 nM to 1000 nM, 1000 nM to 10 μM, 10 μM to 100 μM, or 100 μM to 1000 μM. In some embodiments, the cell is contacted with an oligonucleotide, a nanostructure, or a mASO-SNA described herein at a concentration of 0.001 nM, 0.01 nM, 0.1 nM, 1 nM, 10 nM, 100 nM, 1000 nM, 10 μM, 100 μM, 1000 μM or above 1000 μM.

The oligonucleotides described herein can be used alone or in conjugates with other molecules such as detection or cytotoxic agents in the detection and treatment methods of the invention, as described in more detail herein.

The oligonucleotide may be, for instance, coupled or conjugated to a detectable label. A detectable label is a moiety, the presence of which can be ascertained directly or indirectly. Generally, detection of the label involves an emission of energy by the label. The label can be detected directly by its ability to emit and/or absorb photons or other atomic particles of a particular wavelength (e.g., radioactivity, luminescence, optical or electron density, etc.). A label can be detected indirectly by its ability to bind, recruit and, in some cases, cleave another moiety which itself may emit or absorb light of a particular wavelength (e.g., epitope tag such as the FLAG epitope, enzyme tag such as horseradish peroxidase, etc.). An example of indirect detection is the use of a first enzyme label which cleaves a substrate into visible products. The label may be of a chemical, peptide or nucleic acid molecule nature although it is not so limited. Other detectable labels include radioactive isotopes such as P³² or H³, luminescent markers such as fluorochromes, optical or electron density markers, etc., or epitope tags such as the FLAG epitope or the HA epitope, biotin, avidin, and enzyme tags such as horseradish peroxidase, β-galactosidase, etc. The label may be bound to an oligonucleotide during or following its synthesis. There are many different labels and methods of labeling known to those of ordinary skill in the art. Examples of the types of labels that can be used in the present invention include enzymes, radioisotopes, fluorescent compounds, colloidal metals, chemiluminescent compounds, and bioluminescent compounds. Those of ordinary skill in the art will know of other suitable labels for the oligonucleotides described herein, or will be able to ascertain such, using routine experimentation. Furthermore, the coupling or conjugation of these labels to the oligonucleotides of the invention can be performed using standard techniques common to those of ordinary skill in the art.

Conjugation of the oligonucleotides to a detectable label facilitates, among other things, the use of such agents in diagnostic assays. Another category of detectable labels includes diagnostic and imaging labels (generally referred to as in vivo detectable labels) such as for example magnetic resonance imaging (MRI): Gd(DOTA); for nuclear medicine: ²⁰¹Tl, gamma-emitting radionuclide 99mTc; for positron-emission tomography (PET): positron-emitting isotopes, (18)F-fluorodeoxyglucose ((18)FDG), (18)F-fluoride, copper-64, gadodiamide, and radioisotopes of Pb(II) such as 203Pb; 111In. In such instances, the use of the oligonucleotide could be observed as the oligonucleotide provides an antisense effect.

The conjugations or modifications described herein employ routine chemistry, which chemistry does not form a part of the invention and which chemistry is well known to those skilled in the art of chemistry. The use of protecting groups and known linkers such as mono- and hetero-bifunctional linkers are well documented in the literature and will not be repeated here.

As used herein, “conjugated” means two entities stably bound to one another by any physiochemical means. It is important that the nature of the attachment is such that it does not impair substantially the effectiveness of either entity. Keeping these parameters in mind, any covalent or non-covalent linkage known to those of ordinary skill in the art may be employed. In some embodiments, covalent linkage is preferred. Noncovalent conjugation includes hydrophobic interactions, ionic interactions, high affinity interactions such as biotin-avidin and biotin-streptavidin complexation and other affinity interactions. Such means and methods of attachment are well known to those of ordinary skill in the art. A variety of methods may be used to detect the label, depending on the nature of the label and other assay components.

Pharmaceutical compositions of the present invention comprise an effective amount of one or more agents, dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. Moreover, for animal (e.g., human) administration, it will be understood that preparations should meet sterility, pyrogenicity, general safety and purity standards as required by FDA Office of Biological Standards. The compounds are generally suitable for administration to humans. This term requires that a compound or composition be nontoxic and sufficiently pure so that no further manipulation of the compound or composition is needed prior to administration to humans.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art.

The agent may comprise different types of carriers depending on whether it is to be administered in solid, liquid, gel, cream, or aerosol form, and whether it need to be sterile for such routes of administration as injection. The present invention can be administered intravenously, intradermally, intraarterially, intralesionally, intratumorally, intracranially, intrathecally, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, via eyedrops, orally, topically, locally, inhalation (e.g., aerosol inhalation), injection, infusion, continuous infusion, localized perfusion bathing target cells directly, via a catheter, via a lavage, in creams, in gel, in lipid compositions (e.g., liposomes), or by other method or any combination of the forgoing as would be known to one of ordinary skill in the art.

In any case, the composition may comprise various antioxidants to retard oxidation of one or more components. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The agent may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups also can be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

The compounds of the invention may be administered directly to a tissue. Direct tissue administration may be achieved by direct injection, topical application, or local application. The compounds may be administered once, or alternatively they may be administered in a plurality of administrations. If administered multiple times, the compounds may be administered via different routes. For example, the first (or the first few) administrations may be made directly into the affected tissue while later administrations may be systemic.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

According to the methods of the invention, the compound may be administered in a pharmaceutical composition. In general, a pharmaceutical composition comprises the compound of the invention and a pharmaceutically-acceptable carrier. As used herein, a pharmaceutically-acceptable carrier means a non-toxic material that does not interfere with the effectiveness of the biological activity of the active ingredients.

Pharmaceutically acceptable carriers include diluents, fillers, salts, buffers, stabilizers, solubilizers and other materials which are well-known in the art. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The compounds of the invention may be formulated into preparations in solid, semi-solid, liquid or gaseous forms such as tablets, capsules, powders, granules, ointments, solutions, depositories, inhalants and injections, and usual ways for oral, parenteral or surgical administration. The invention also embraces pharmaceutical compositions which are formulated for local administration, such as by implants.

Compositions suitable for oral administration may be presented as discrete units, such as capsules, tablets, lozenges, each containing a predetermined amount of the active agent. Other compositions include suspensions in aqueous liquids or non-aqueous liquids such as a syrup, elixir or an emulsion.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, thermoreversible hydrogels such as pluronic F-127, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. Techniques for preparing aerosol delivery systems are well known to those of skill in the art.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compounds.

The compositions of the invention may be formulated in a topical composition for administration to the skin or a body cavity. Suitable topical vehicles and vehicle components are well known in the cosmetic and pharmaceutical arts, and include such vehicles (or vehicle components) as water; thermoreversible hydrogels such as pluronic F-127, organic solvents such as alcohols (particularly lower alcohols readily capable of evaporating from the skin such as ethanol), glycols (such as propylene glycol, butylene glycol, and glycerin), aliphatic alcohols (such as lanolin); mixtures of water and organic solvents (such as water and alcohol), and mixtures of organic solvents such as alcohol and glycerin (optionally also with water); lipid-based materials such as fatty acids, acylglycerols (including oils, such as mineral oil, and fats of natural or synthetic origin), phosphoglycerides, sphingolipids and waxes; protein-based materials such as collagen and gelatin; silicone-based materials (both non-volatile and volatile) such as cyclomethicone, demethiconol and dimethicone copolyol (Dow Corning); hydrocarbon-based materials such as petrolatum and squalane; anionic, cationic and amphoteric surfactants and soaps; sustained-release vehicles such as microsponges and polymer matrices; stabilizing and suspending agents; emulsifying agents; and other vehicles and vehicle components that are suitable for administration to the skin, as well as mixtures of topical vehicle components as identified above or otherwise known to the art. The vehicle may further include components adapted to improve the stability or effectiveness of the applied formulation, such as preservatives, antioxidants, skin penetration enhancers, sustained release materials, and the like.

The choice of a suitable vehicle will depend on the particular physical form and mode of delivery that the formulation is to achieve. Examples of suitable forms include liquids (e.g., gargles and mouthwashes, including dissolved forms of the strontium cation as well as suspensions, emulsions and the like); solids and semisolids such as gels, foams, pastes, creams, ointments, “sticks” (as in lipsticks or underarm deodorant sticks), powders and the like; formulations containing liposomes or other delivery vesicles; rectal or vaginal suppositories, creams, foams, gels or ointments; and other forms. Typical modes of delivery include application using the fingers; application using a physical applicator such as a cloth, tissue, swab, stick or brush (as achieved for example by soaking the applicator with the formulation just prior to application, or by applying or adhering a prepared applicator already containing the formulation—such as a treated or premoistened bandage, wipe, washcloth or stick-to the skin); spraying (including mist, aerosol or foam spraying); dropper application (as for example with ear drops); sprinkling (as with a suitable powder form of the formulation); and soaking.

Topical formulations also include formulations for rectal and vaginal administration. Formulations for rectal administration may be presented as a suppository with a suitable base comprising, for example, cocoa butter. Formulations suitable for vaginal administration may be presented as tablets, pessaries, tampons, creams, gels, pastes, foams or spray formulations containing in addition to the active ingredient such carriers as are known in the art to be appropriate.

In yet other embodiments, a delivery vehicle is a biocompatible microparticle or implant that is suitable for implantation into the mammalian recipient. Other delivery systems can include time-release, delayed release or sustained release delivery systems. Such systems can avoid repeated administrations of the compound, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides.

In some embodiments the antisense nucleic acids of the invention are formulated as a stable self-assembling nanostructure. The nanostructure includes a IL1B antisense oligonucleotide, wherein the antisense oligonucleotide is associated with a core. The core may be a solid or a hollow core, such as a liposomal core. A solid core is a spherical shaped material that does not have a hollow center. The term spherical as used herein refers to a general shape and does not imply or is not limited to a perfect sphere or round shape. It may include imperfections.

Solid cores can be constructed from a wide variety of materials known to those skilled in the art including but not limited to: noble metals (gold, silver), transition metals (iron, cobalt) and metal oxides (silica). In addition, these cores may be inert, paramagnetic, or superparamagnetic. These solid cores can be constructed from either pure compositions of described materials, or in combinations of mixtures of any number of materials, or in layered compositions of materials. In addition, solid cores can be composed of a polymeric core such as amphiphilic block copolymers, hydrophobic polymers such as polystyrene, poly(lactic acid), poly(lactic co-glycolic acid), poly(glycolic acid), poly(caprolactone) and other biocompatible polymers known to those skilled in the art.

The core may alternatively be a hollow core, which has at least some space in the center region of a shell material. Hollow cores include liposomal cores and niosomes. A liposomal core as used herein refers to a centrally located core compartment formed by a component of the lipids or phospholipids that form a lipid bilayer. “Liposomes” are artificial, self closed vesicular structure of various sizes and structures, where one or several membranes encapsulate an aqueous core. Most typically liposome membranes are formed from lipid bilayers membranes, where the hydrophilic head groups are oriented towards the aqueous environment and the lipid chains are embedded in the lipophilic core. Liposomes can be formed as well from other amphiphilic monomeric and polymeric molecules, such as polymers, like block copolymers, or polypeptides. Unilamellar vesicles are liposomes defined by a single membrane enclosing an aqueous space. In contrast, oligo- or multilamellar vesicles are built up of several membranes. Typically, the membranes are roughly 4 nm thick and are composed of amphiphilic lipids, such as phospholipids, of natural or synthetic origin. Optionally, the membrane properties can be modified by the incorporation of other lipids such as sterols or cholic acid derivatives.

The lipid bilayer is composed of two layers of lipid molecules. Each lipid molecule in a layer is oriented substantially parallel to adjacent lipid bilayers, and two layers that form a bilayer have the polar ends of their molecules exposed to the aqueous phase and the non-polar ends adjacent to each other. The central aqueous region of the liposomal core may be empty or filled fully or partially with water, an aqueous emulsion, oligonucleotides, or other therapeutic or diagnostic agents.

Niosomes are vesicles formed from non-ionic surfactant oriented in a bilayer. Niosomes commonly have cholesterol added as an excipient, but other lipid-based and non-lipid-based constituents can also be included. Methods for preparation of niosomes are known in the art. In some embodiments polyethylene glycol (PEG) is included during or following niosome preparation. Niosome vesicles are structurally and functionally analogous to liposomes, but are based on non-ionic surfactant rather than lipid as the primary constiuent. Common non-ionic surfactants used include sorbitans (spans) or polysorbates (tween); however, a wide variety of non-ionic surfactants can be used to prepare niosomes.

“Lipid” refers to its conventional sense as a generic term encompassing fats, lipids, alcohol-ether-soluble constituents of protoplasm, which are insoluble in water. Lipids usually consist of a hydrophilic and a hydrophobic moiety. In water lipids can self organize to form bilayers membranes, where the hydrophilic moieties (head groups) are oriented towards the aqueous phase, and the lipophilic moieties (acyl chains) are embedded in the bilayers core. Lipids can comprise as well two hydrophilic moieties (bola amphiphiles). In that case, membranes may be formed from a single lipid layer, and not a bilayer. Typical examples for lipids in the current context are fats, fatty oils, essential oils, waxes, steroid, sterols, phospholipids, glycolipids, sulpholipids, aminolipids, chromolipids, and fatty acids. The term encompasses both naturally occurring and synthetic lipids. Preferred lipids in connection with the present invention are: steroids and sterol, particularly cholesterol, phospholipids, including phosphatidyl, phosphatidylcholines and phosphatidylethanolamines and sphingomyelins. Where there are fatty acids, they could be about 12-24 carbon chains in length, containing up to 6 double bonds. The fatty acids are linked to the backbone, which may be derived from glycerol. The fatty acids within one lipid can be different (asymmetric), or there may be only 1 fatty acid chain present, e.g. lysolecithins. Mixed formulations are also possible, particularly when the non-cationic lipids are derived from natural sources, such as lecithins (phosphatidylcholines) purified from egg yolk, bovine heart, brain, liver or soybean.

The liposomal core can be constructed from one or more lipids known to those in the art including but not limited to: 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), and 1,2-dihexadecanoyl-sn-glycero-3-phosphoethanolamine (DPPE), sphingolipids such as sphingosine, sphingosine phosphate, methylated sphingosines and sphinganines, ceramides, ceramide phosphates, 1-0 acyl ceramides, dihydroceramides, 2-hydroxy ceramides, sphingomyelin, glycosylated sphingolipids, sulfatides, gangliosides, phosphosphingolipids, and phytosphingosines of various lengths and saturation states and their derivatives, phospholipids such as phosphatidylcholines, lysophosphatidylcholines, phosphatidic acids, lysophosphatidic acids, cyclic LPA, phosphatidylethanolamines, lysophosphatidylethanolamines, phosphatidylglycerols, lysophosphatidylglycerols, phosphatidylserines, lysophosphatidylserines, phosphatidylinositols, inositol phosphates, LPI, cardiolipins, lysocardiolipins, bis(monoacylglycero) phosphates, (diacylglycero) phosphates, ether lipids, diphytanyl ether lipids, and plasmalogens of various lengths, saturation states, and their derivatives, sterols such as cholesterol, desmosterol, stigmasterol, lanosterol, lathosterol, diosgenin, sitosterol, zymosterol, zymostenol, 14-demethyl-lanosterol, cholesterol sulfate, DHEA, DHEA sulfate, 14-demethyl-14-dehydrlanosterol, sitostanol, campesterol, ether anionic lipids, ether cationic lipids, lanthanide chelating lipids, A-ring substituted oxysterols, B-ring substituted oxysterols, D-ring substituted oxysterols, side-chain substituted oxysterols, double substituted oxysterols, cholestanoic acid derivatives, fluorinated sterols, fluorescent sterols, sulfonated sterols, phosphorylated sterols, and polyunsaturated sterols of different lengths, saturation states, and their derivatives.

In certain embodiments, the diameter of the core is from 1 nm to about 250 nm in mean diameter, about 1 nm to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, or about 1 nm to about 20 nm in mean diameter, or about 1 nm to about 10 nm in mean diameter.

The oligonucleotides may be positioned on the exterior of the core, within the walls of the core and/or in the center of the core. An oligonucleotide that is positioned on the core is typically referred to as coupled to the core. Coupled may be direct or indirect. In some embodiments at least 5, 10, 15, 25, 50, 75, 100, 200, 300, 400, 500, 600, 700, 800, 900, 1000, 5000 or 10,000 oligonucleotides or any range combination thereof are on the exterior of the core. In some embodiments, 1-1000, 10-500, 50-250, or 50-300 oligonucleotides are present on the surface.

The oligonucleotides of the oligonucleotide shell may be oriented in a variety of directions. In some embodiments the oligonucleotides are oriented radially outwards. The orientation of these oligonucleotides can be either 5′ distal/3′ terminal in relation to the core, or 3′ distal/5′terminal in relation to the core, or laterally oriented around the core. In one embodiment one or a multiplicity of different oligonucleotides are present on the same surface of a single SNA. In all cases, at least 1 oligonucleotide is present on the surface but up to 10,000 can be present.

The oligonucleotides may be linked to the core or to one another and/or to other molecules such an active agents either directly or indirectly through a linker. The oligonucleotides may be conjugated to a linker via the 5′ end or the 3′ end, e.g. [Sequence, 5′-3′]-Linker or Linker-[Sequence, 5′-3′]. Some or all of the oligonucleotides of the nanostructure may be linked to one another either directly or indirectly through a covalent or non-covalent linkage. The linkage of one oligonucleotide to another oligonucleotide may be in addition to or alternatively to the linkage of that oligonucleotide to liposomal core.

The oligonucleotide shell may be anchored to the surface of the core through one or multiple of linker molecules, including but not limited to: any chemical structure containing one or multiple thiols, such as the various chain length alkane thiols, cyclic dithiol, lipoic acid, or other thiol linkers known to those skilled in the art.

In an embodiment containing a liposomal core, the oligonucleotide shell may be anchored to the surface of the liposomal core through conjugation to one or a multiplicity of linker molecules including but not limited to: tocopherols, sphingolipids such as sphingosine, sphingosine phosphate, methylated sphingosines and sphinganines, ceramides, ceramide phosphates, 1-0 acyl ceramides, dihydroceramides, 2-hydroxy ceramides, sphingomyelin, glycosylated sphingolipids, sulfatides, gangliosides, phosphosphingolipids, and phytosphingosines of various lengths and saturation states and their derivatives, phospholipids such as phosphatidylcholines, lysophosphatidylcholines, phosphatidic acids, lysophosphatidic acids, cyclic LPA, phosphatidylethanolamines, lysophosphatidylethanolamines, phosphatidylglycerols, lysophosphatidylglycerols, phosphatidylserines, lysophosphatidylserines, phosphatidylinositols, inositol phosphates, LPI, cardiolipins, lysocardiolipins, bis(monoacylglycero) phosphates, (diacylglycero) phosphates, ether lipids, diphytanyl ether lipids, and plasmalogens of various lengths, saturation states, and their derivatives, sterols such as cholesterol, desmosterol, stigmasterol, lanosterol, lathosterol, diosgenin, sitosterol, zymosterol, zymostenol, 14-demethyl-lanosterol, cholesterol sulfate, DHEA, DHEA sulfate, 14-demethyl-14-dehydrlanosterol, sitostanol, campesterol, ether anionic lipids, ether cationic lipids, lanthanide chelating lipids, A-ring substituted oxysterols, B-ring substituted oxysterols, D-ring substituted oxysterols, side-chain substituted oxysterols, double substituted oxysterols, cholestanoic acid derivatives, fluorinated sterols, fluorescent sterols, sulfonated sterols, phosphorylated sterols, and polyunsaturated sterols of different lengths, saturation states, and their derivatives.

The oligonucleotide may also be associated with the core by being embedded within the core (liposomal core) or it may be attached or linked, either indirectly (i.e. non-covalently or covalently through other molecules such a linkers) or directly (i.e. covalently).

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

In order that the invention described herein may be more fully understood, the following examples are set forth. The examples described in this application are offered to illustrate the compounds, pharmaceutical compositions, and methods provided herein and are not to be construed in any way as limiting their scope.

### EXAMPLES

### Example 1. Selection of IL-1 Beta Targeting SNA Compounds

### Methods and Materials

### Cell Culture

Human neonatal foreskin keratinocytes (HFKs) were cultured in M154 media (Life Technologies) supplemented with Human Keratinocyte Growth Supplement (Life Technologies), 0.07 mM CaCl₂, 10 μg/mL gentamicin, and 0.25 μg/mL amphotericin B. Cells were maintained at 37° C. in a 5% CO₂ humidified incubator.

### SNA Synthesis

The SNA construct is a 3-dimensional arrangement of oligonucleotides where the nucleic acids are densely packed and radially oriented. The dense and radial arrangement of the oligonucleotides is driven by hydrophobic interactions between the cholesterol moieties at the end of the nucleic acid chains and the membrane of a 20 nm diameter liposome. The oligonucleotides and liposomes assemble spontaneously in aqueous solutions at a wide range of concentrations, thereby producing the SNA construct.

The oligonucleotides were synthesized at the 2 mole scale employing standard UniLinker support (ChemGenes). The DNA, 2′-O-Me RNA monomers and hexa(ethylene glycol) spacers were obtained from ChemGenes Corporation. The cholesterol modifier was obtained from Glen Research. Linkages were either standard phosphodiesters or phosphorothioates prepared with a solution of 0.2 M phenylacetyl disulfide (PADS) in a mixture of 1:1 lutidine:ACN. Synthesis was performed DMT-off, in the 3′ to 5′ direction. After synthesis, the oligonucleotide was cleaved from the support and de-protected using a 4:1 mixture of ammonium hydroxide and ethanol at 55° C. for 16 hours. The oligonucleotide was purified via high performance liquid chromatography (HPLC) techniques. Molecular weights and extinction coefficients were calculated using the IDT OligoAnalyzer. The verification of the oligonucleotide product molecular weights was performed using electrospray ionization mass spectrometry (ESI-MS). Finally, the oligonucleotide concentrations were determined by UV-absorbance at 260 nm on a microplate reader (BioTek).

Liposomes were formulated by first dissolving 250 mg 1,2-dioleoyl-sn-glycero-3-phosphocholine (DOPC) in chloroform to a final concentration of 50 mg/mL. The solvent was then removed under nitrogen to form a thin lipid film. The film was lyophilized overnight to ensure all solvent was removed. The lipid film was subsequently hydrated with 10 mM phosphate buffered saline (PBS) and sonication/freeze-fracture were used to form large, unilamellar vesicles. These vesicles were then continuously homogenized through a micro-fluidizer at up to 25 kpsi, until the desired mean number diameter of 20 nm was achieved. Liposome concentrations were determined using a choline quantification assay and the particle size and dispersion were measured by dynamic light scattering (DLS).

SNAs targeting human IL1B were formulated by mixing a 30 fold molar excess of cholesterol-modified oligonucleotides (Table 1) to the liposome suspension in PBS followed by an overnight incubation at 4° C. Similarly, a control SNA was formulated using a non-complimentary control antisense sequence (confirmed by NCBI Blast), comprising the same ‘gap-mer’ design and 3′ chemical modifications. Oligonucleotide sequences for all compounds synthesized and tested are shown in table 6.

### SNA Treatment

HFKs were seeded in 96-well, tissue culture plates at a cell density of 5,000-15,000 cells per well. Cells were allowed to rest in the incubator 1-3 days following plating until cells were ˜75% confluent and then were treated in triplicate with either an IL1B targeted antisense SNA or a non-complimentary control (confirmed by NCBI Blast) SNA, at concentrations of 10000, 1000, 100, and 10 nM in fresh maintenance media.

RNA Extraction and qRT-PCR

Cells were lysed in RLT Buffer (Qiagen) at 24 hours post-transfection. RNA was isolated from lysates using the RNEasy 96-well kit (Qiagen) according to the manufacturer's instructions. cDNA was then synthesized from RNA isolates using the cDNA high capacity reverse transcription kit (Life Technologies). cDNA was prepared on a thermocycler with the following temperature program: 25° C. for 10 minutes, 37° C. for 90 minutes, 85° C. for 5 minutes followed by a 4° C. hold. The resulting cDNA was diluted 8 fold with nuclease-free water. qPCR was performed using 6 μL of the diluted cDNA, 4.66 μL LightCycler480 Probes Master Mix (Roche), 0.47 μL human IL1B specific FAM-labeled probe and primers, and 0.37 μL human Glyceraldehyde-3-phosphate dehydrogenase (GAPDH) specific HEX-labeled probe and primers per reaction well of a 384-well optical reaction plate (Roche). The primer and probe set for IL1B was purchased commercially (Hs01555410_ml, Thermo Fisher Scientific). The primer and probe set for GAPDH was designed using the known human genome sequence (NCBI reference sequences NM_002046.5 (SEQ ID NO: 145)) and was found to be specific by “blastn” analysis (NCBI). The oligonucleotide sequences used for GAPDH were: forward 5′-CAA GGT CAT CCA TGA CAA CTT TG-3′ (SEQ ID NO: 142), reverse 5′-GGG CCA TCC ACA GTC TTC T-3′ (SEQ ID NO: 143), probe 5′-HEX-ACC ACA GTC CAT GCC ATC ACT GCC A-BHQ1-3′ (SEQ ID NO: 144). qPCR reactions, in technical duplicate, were carried out on the Roche Lightcycler 480 under the following conditions: initial denaturation at 95° C. for 10 minutes and then 50 cycles of denaturation at 95° C. for 10 seconds, annealing at 60° C. for 30 seconds and extension at 72° C. for 1 second. Cp values were obtained by analysis with the 2nd derivative method. Relative gene expression was determined by normalization with the housekeeping gene (GAPDH) and the ΔΔ-Ct method.

TABLE 1
Oligonucleotide Compounds.

| Compound | Antisense Oligonucleotide |
|---|---|
| ID | Sequence (5′ to 3′) |
| hIL1B_178 | mGmAmAmGmGmAG*C*A*C*T*T*mCmAmUmCmUmG/ |
| | isp18//isp18//3CholTEG/ (SEQ ID NO: 6) |
| hIL1B_599 | mCmCmAmAmGmGC*C*A*C*A*G*mGmUmAmUmUmU/ |
| | isp18//isp18//3CholTEG/ (SEQ ID NO: 7) |
| hIL1B_750 | mUmCmCmAmGmCT*T*G*T*T*A*mUmUmGmAmUmU/ |
| | isp18//isp18//3CholTEG/ (SEQ ID NO: 10) |
| hIL1B_1040 | mAmGmAmUmCmCT*C*T*T*A*G*mCmAmCmUmAmC/ |
| | isp18//isp18//3CholTEG/ (SEQ ID NO: 3) |
| hIL1B_241- | mGmUmAmGmUmGG*T*G*G*T*/iMe- |
| 12m5C | dC/*mGmGmAmGmAmU/isp18//isp18//3CholTEG/ |
| | (SEQ ID NO: 43) |

“*” denotes a phosphorothioate bond, “m” denotes an 2′-O-methylated base, “/iSp18/” denotes a hexa(ethylene glycol) spacer, “/3CholTEG/” denotes a 3′ tri(ethylene glycol) bound to a cholesteryl ester.

### Results

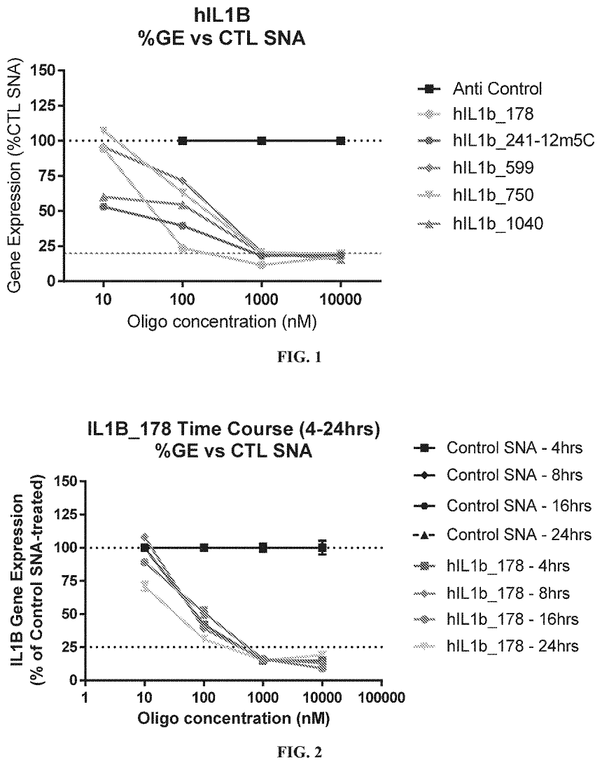

Of the screened compounds, five IL1B mRNA targeted SNAs were selected for follow-up. These compounds showed dose-dependent knockdown of IL1B mRNA in human primary keratinocytes after 24 hours of treatment and maximum knockdown of >70% (FIG. 1). Knockdown was observed when compared to both untreated cells and control SNA.

### Discussion and Conclusions

It has been shown here that when the IL1B mRNA targeted SNAs are applied to human primary keratinocytes, there is a dose dependent decrease of the target mRNA expression with greater targeted SNA concentrations. Given the importance of IL-1β signaling in inflammation, anti-IL1B SNAs could be valuable compounds in treating inflammatory conditions.

TABLE 2
Percent Expression of IL-1 beta mRNA at 10 μM vs
CTL SNA for all compounds screened in this experiment.
Average of two screens and standard deviation shown.

| | | % Average | |
|---|---|---|---|
| | | IL1b | Standard |
| SEQ ID NO: | Compound ID | Expression | Deviation |
| 2 | hIL1b_233 | 13.5 | 7.95 |
| 3 | hIL1b_1040 | 15.2 | 4.69 |
| 4 | hIL1b_241 | 15.6 | 3.43 |
| 5 | hIL1b_863 | 16.8 | 0.3 |
| 6 | hIL1b_178 | 17.2 | 2.02 |
| 7 | hIL1b_599 | 16.5 | 8.53 |
| 8 | hIL1b_1170 | 16.6 | 5.49 |
| 9 | hIL1b_g8196 | 17.3 | 5.83 |
| 10 | hIL1b_750 | 19.4 | 5.12 |
| 11 | hIL1b_864 | 22.5 | 2.88 |
| 12 | hIL1b_397 | 22.8 | 6.31 |
| 13 | hIL1b_235 | 25.4 | 12.07 |
| 14 | hIL1b_242 | 26.2 | 0.43 |
| 15 | hIL1b_277 | 27.3 | 3 |
| 16 | hIL1b_1409 | 27.3 | 18.57 |
| 17 | hIL1b_805 | 29.7 | 14.26 |
| 18 | hIL1b_748 | 32.5 | 12.13 |
| 19 | hIL1b_g8398 | 33.4 | 9.05 |
| 20 | hIL1b_473 | 38.8 | 26.91 |
| 21 | hIL1b_1412 | 40.8 | 4.89 |
| 22 | hIL1b_1145-5MdzG | 45.6 | 10.61 |
| 23 | hIL1b_g5527 | 47.5 | 17.93 |
| 24 | hIL1b_1211-14MdzG | 49.7 | 12.5 |
| 25 | hIL1b_g5589 | 50.1 | 4.91 |
| 26 | hIL1b_1231-9dzG | 53.3 | 16.05 |
| 27 | hIL1b_813-4MdzG | 58.2 | 25.1 |
| 28 | hIL1b_693-5,16MdzG | 58.4 | 15.45 |
| 29 | hIL1b_126 | 59.5 | 15.89 |
| 30 | hIL1b_698-10dzG | 62.1 | 28.68 |
| 31 | hIL1b_g8945 | 67.5 | 21.45 |
| 32 | hIL1b_1208-7dzG | 67.8 | 25.1 |
| 33 | hIL1b_545 | 69.3 | 3.79 |
| 34 | hIL1b_1229-7dzG | 71.2 | 28.24 |
| 35 | hIL1b_1207-10dzG | 74.2 | 22.14 |
| 36 | hIL1b_1186-5MdzG | 76.1 | 21.13 |
| 37 | hIL1b_1230-8dzG | 79.5 | 8.36 |
| 38 | hIL1b_1185-4MdzG | 83 | 0.17 |
| 39 | hIL1b_g6154 | 86.5 | 23.58 |
| 40 | hIL1b_g10480 | 105.7 | 25.47 |
| 41 | hIL1b_g11198 | 112.4 | 40.65 |
| 42 | hIL1b_676 | 123.2 | 77.44 |
| 43 | hIL1B_241-12m5C | 18.5 | 2.12 |

### Example 2. It is Demonstrated that Knockdown of Human IL1B mRNA (GeneBank Accession Number NM_000576, SEQ ID NO: 1) in Primary Human Keratinocytes by SNAs Begins at ≤4 Hours after Initiating Treatment

### Methods and Materials

### Cell Culture

Human neonatal foreskin keratinocytes (HFKs) were cultured in M154 media (Life Technologies) supplemented with Human Keratinocyte Growth Supplement (Life Technologies), 0.07 mM CaCl₂, 10 μg/mL gentamicin, and 0.25 μg/mL amphotericin B. Cells were maintained at 37° C. in a 5% CO₂ humidified incubator.

### SNA Synthesis

The SNA construct is a 3-dimensional arrangement of oligonucleotides where the nucleic acids are densely packed and radially oriented. The dense and radial arrangement of the oligonucleotides is driven by hydrophobic interactions between the cholesterol moieties at the end of the nucleic acid chains and the membrane of a 20 nm diameter liposome. The oligonucleotides and liposomes assemble spontaneously in aqueous solutions at a wide range of concentrations, thereby producing the SNA construct.

The oligonucleotides were synthesized at the 2 mole scale employing standard UniLinker support (ChemGenes). The DNA, 2′-O-Me RNA monomers and hexa(ethylene glycol) spacers were obtained from ChemGenes Corporation. The cholesterol modifier was obtained from Glen Research. Linkages were either standard phosphodiesters or phosphorothioates prepared with a solution of 0.2 M phenylacetyl disulfide (PADS) in a mixture of 1:1 lutidine:ACN. Synthesis was performed DMT-off, in the 3′ to 5′ direction. After synthesis, the oligonucleotide was cleaved from the support and de-protected using a 4:1 mixture of ammonium hydroxide and ethanol at 55° C. for 16 hours. The oligonucleotide was purified via high performance liquid chromatography (HPLC) techniques. Molecular weights and extinction coefficients were calculated using the IDT OligoAnalyzer. The verification of the oligonucleotide product molecular weights was performed using electrospray ionization mass spectrometry (ESI-MS). Finally, the oligonucleotide concentrations were determined by UV-absorbance at 260 nm on a microplate reader (BioTek).

Liposomes were formulated by first dissolving 250 mg 1,2-dioleoyl-sn-glycero-3-phosphocholine (DOPC) in chloroform to a final concentration of 50 mg/mL. The solvent was then removed under nitrogen to form a thin lipid film. The film was lyophilized overnight to ensure all solvent was removed. The lipid film was subsequently hydrated with 10 mM phosphate buffered saline (PBS) and sonication/freeze-fracture were used to form large, unilamellar vesicles. These vesicles were then continuously homogenized through a micro-fluidizer at up to 25 kpsi, until the desired mean number diameter of 20 nm was achieved. Liposome concentrations were determined using a choline quantification assay and the particle size and dispersion were measured by dynamic light scattering (DLS).

SNAs targeting human IL1B were formulated by mixing a 30 fold molar excess of cholesterol-modified oligonucleotides to the liposome suspension in PBS followed by an overnight incubation at 4° C. Similarly, a control SNA was formulated using a non-complimentary control antisense sequence (confirmed by NCBI Blast), comprising the same ‘gap-mer’ design and 3′ chemical modifications.

### SNA Treatment

HFKs were seeded in 96-well, tissue culture plates at a cell density of 4,000 cells per well. Cells were allowed to rest in the incubator for 3 days following plating and then were treated in triplicate with either an IL1B targeted antisense SNA or a non-complimentary control (confirmed by NCBI Blast) SNA, at concentrations of 10000, 1000, 100, and 10 nM in fresh maintenance media.

RNA Extraction and qRT-PCR

Cells were lysed in RLT Buffer (Qiagen) at 4-24 hours post-transfection, as indicated. RNA was isolated from lysates using the RNEasy 96-well kit (Qiagen) according to the manufacturer's instructions. cDNA was then synthesized from RNA isolates using the cDNA high capacity reverse transcription kit (Life Technologies). cDNA was prepared on a thermocycler with the following temperature program: 25° C. for 10 minutes, 37° C. for 90 minutes, 85° C. for 5 minutes followed by a 4° C. hold. The resulting cDNA was diluted 8 fold with nuclease-free water. qPCR was performed using 6 μL of the diluted cDNA, 4.66 μL LightCycler480 Probes Master Mix (Roche), 0.47 μL human IL1B specific FAM-labeled probe and primers, and 0.37 μL human Glyceraldehyde-3-phosphate dehydrogenase (GAPDH) specific HEX-labeled probe and primers per reaction well of a 384-well optical reaction plate (Roche). The primer and probe set for IL1B was purchased commercially (Hs01555410_m1, Thermo Fisher Scientific). The primer and probe set for GAPDH was designed using the known human genome sequence (NCBI reference sequences NM_002046.5 (SEQ ID NO: 145)) and was found to be specific by “blastn” analysis (NCBI). The oligonucleotide sequences used for GAPDH were: forward 5′-CAA GGT CAT CCA TGA CAA CTT TG-3′ (SEQ ID NO: 142), reverse 5′-GGG CCA TCC ACA GTC TTC T-3′ (SEQ ID NO: 143), probe 5′-HEX-ACC ACA GTC CAT GCC ATC ACT GCC A-BHQ1-3′ (SEQ ID NO: 144). qPCR reactions, in technical duplicate, were carried out on the Roche Lightcycler 480 under the following conditions: initial denaturation at 95° C. for 10 minutes and then 50 cycles of denaturation at 95° C. for 10 seconds, annealing at 60° C. for 30 seconds and extension at 72° C. for 1 second. Cp values were obtained by analysis with the 2nd derivative method. Relative gene expression was determined by normalization with the housekeeping gene (GAPDH) and the ΔΔ-Ct method.

TABLE 3
Oligonucleotide Compound.

| Compound | Oligonucleotide Sequence (5′ to 3') |
|---|---|
| hIL1B_178 | mGmAmAmGmGmAG*C*A*C*T*T*mCmAmUmCmUmG/ |
| | isp18//isp18//3CholTEG/ |
| | (SEQ ID NO: 6) |

“*” denotes a phosphorothioate bond, “m” denotes an 2′-O-methylated base, “/iSp18/” denotes a hexa(ethylene glycol) spacer, “/3CholTEG/” denotes a 3′ tri(ethylene glycol) bound to a cholesteryl ester.

### Results

As seen in FIG. 2, at higher concentrations, IL1B mRNA knockdown was equivalent after 4-24 hours of treatment. At lower concentrations, the majority of the knockdown was apparent by 4 hrs of treatment, but maximal knockdown at those concentrations was not achieved until 24 hours of treatment.

### Discussion and Conclusions

When treatment is performed at higher concentrations (1-10 μM), the level of IL1B knockdown is equivalent at each time point tested from 4 through 24 hours. At lower concentrations (10-100 nM), the knockdown is similar at 4-16 hours of treatment but increases somewhat by 24 hours of treatment. These data indicate that SNAs produce rapid onset of target mRNA knockdown, beginning at ≤4 hours after initiating treatment.

### Example 3. It is Demonstrated that Knockdown of Human IL1B mRNA (GeneBank Accession Number NM_000576, SEQ ID NO: 1) by SNAs in Primary Human Keratinocytes Persists ≥4 Days after Initiating Treatment

### Methods and Materials

### Cell Culture

Human neonatal foreskin keratinocytes (HFKs) were cultured in M154 media (Life Technologies) supplemented with bovine pituitary extract (ThermoFisher). Cells were maintained at 37° C. in a 5% CO₂ humidified incubator.

### SNA Synthesis

The SNA construct is a 3-dimensional arrangement of oligonucleotides where the nucleic acids are densely packed and radially oriented. The dense and radial arrangement of the oligonucleotides is driven by hydrophobic interactions between the cholesterol moieties at the end of the nucleic acid chains and the membrane of a 20 nm diameter liposome. The oligonucleotides and liposomes assemble spontaneously in aqueous solutions at a wide range of concentrations, thereby producing the SNA construct.

The oligonucleotides were synthesized at the 2 μmole scale employing standard UniLinker support (ChemGenes). The DNA, 2′-O-Me RNA monomers and hexa(ethylene glycol) spacers were obtained from ChemGenes Corporation. The cholesterol modifier was obtained from Glen Research. Linkages were either standard phosphodiesters or phosphorothioates prepared with a solution of 0.2 M phenylacetyl disulfide (PADS) in a mixture of 1:1 lutidine:ACN. Synthesis was performed DMT-off, in the 3′ to 5′ direction. After synthesis, the oligonucleotide was cleaved from the support and de-protected using a 4:1 mixture of ammonium hydroxide and ethanol at 55° C. for 16 hours. The oligonucleotide was purified via high performance liquid chromatography (HPLC) techniques. Molecular weights and extinction coefficients were calculated using the IDT OligoAnalyzer. The verification of the oligonucleotide product molecular weights was performed using electrospray ionization mass spectrometry (ESI-MS). Finally, the oligonucleotide concentrations were determined by UV-absorbance at 260 nm on a microplate reader (BioTek).

Liposomes were formulated by first dissolving 250 mg 1,2-dioleoyl-sn-glycero-3-phosphocholine (DOPC) in chloroform to a final concentration of 50 mg/mL. The solvent was then removed under nitrogen to form a thin lipid film. The film was lyophilized overnight to ensure all solvent was removed. The lipid film was subsequently hydrated with 10 mM phosphate buffered saline (PBS) and sonication/freeze-fracture were used to form large, unilamellar vesicles. These vesicles were then continuously homogenized through a micro-fluidizer at up to 25 kpsi, until the desired mean number diameter of 20 nm was achieved. Liposome concentrations were determined using a choline quantification assay and the particle size and dispersion were measured by dynamic light scattering (DLS).

SNAs targeting human IL1B were formulated by mixing a 30 fold molar excess of cholesterol-modified oligonucleotides to the liposome suspension in PBS followed by an overnight incubation at 4° C. Similarly, a control SNA was formulated using a non-complimentary control antisense sequence (confirmed by NCBI Blast), comprising the same ‘gap-mer’ design and 3′ chemical modifications.

### SNA Treatment

HFKs were seeded in 96-well, tissue culture plates at a cell density of 10,000 cells per well. Cells were allowed to rest in the incubator for 3 days following plating and then were treated in triplicate with either an IL1B targeted antisense SNA or a non-complimentary control (confirmed by NCBI Blast) SNA, at 1000 nM concentration in fresh maintenance media. At 24 hours following addition of SNAs, media was removed and replaced with fresh maintenance media.

RNA Extraction and qRT-PCR

Cells were lysed in RLT Buffer (Qiagen) at 1-4 days post-transfection. RNA was isolated from lysates using the RNEasy 96-well kit (Qiagen) according to the manufacturer's instructions. cDNA was then synthesized from RNA isolates using the cDNA high capacity reverse transcription kit (Life Technologies). cDNA was prepared on a thermocycler with the following temperature program: 25° C. for 10 minutes, 37° C. for 90 minutes, 85° C. for 5 minutes followed by a 4° C. hold. The resulting cDNA was diluted 8 fold with nuclease-free water. qPCR was performed using 6 μL of the diluted cDNA, 4.66 μL LightCycler480 Probes Master Mix (Roche), 0.47 μL human IL1B specific FAM-labeled probe and primers, and 0.37 μL human Glyceraldehyde-3-phosphate dehydrogenase (GAPDH) specific HEX-labeled probe and primers per reaction well of a 384-well optical reaction plate (Roche). The primer and probe set for IL1B was purchased commercially (Hs01555410_m1, Thermo Fisher Scientific). The primer and probe set for GAPDH was designed using the known human genome sequence (NCBI reference sequences NM_002046.5 (SEQ ID NO: 145)) and was found to be specific by “blastn” analysis (NCBI). The oligonucleotide sequences used for GAPDH were: forward 5′-CAA GGT CAT CCA TGA CAA CTT TG-3′ (SEQ ID NO: 142), reverse 5′-GGG CCA TCC ACA GTC TTC T-3′ (SEQ ID NO: 143), probe 5′-HEX-ACC ACA GTC CAT GCC ATC ACT GCC A-BHQ1-3′ (SEQ ID NO: 144). qPCR reactions, in technical duplicate, were carried out on the Roche Lightcycler 480 under the following conditions: initial denaturation at 95° C. for 10 minutes and then 50 cycles of denaturation at 95° C. for 10 seconds, annealing at 60° C. for 30 seconds and extension at 72° C. for 1 second. Cp values were obtained by analysis with the 2nd derivative method. Relative gene expression was determined by normalization with the housekeeping gene (GAPDH) and the ΔΔ-Ct method.

TABLE 4
Oligonucleotide Compound.

| | Antisense Oligonucleotide |
|---|---|
| Compound | Sequence (5′ to 3′) |
| SNA 1 | mGmAmAmGmGmAG*C*A*C*T*T*mCmAmUmCmUmG/ |
| (hIL1B_178) | isp18//isp18//3CholTEG/ (SEQ ID NO: 6) |

“*” denotes a phosphorothioate bond, “m” denotes an 2′-O-methylated base, “/iSp18/” denotes a hexa(ethylene glycol) spacer, “/3CholTEG/” denotes a 3′ tri(ethylene glycol) bound to a cholesteryl ester.

### Results

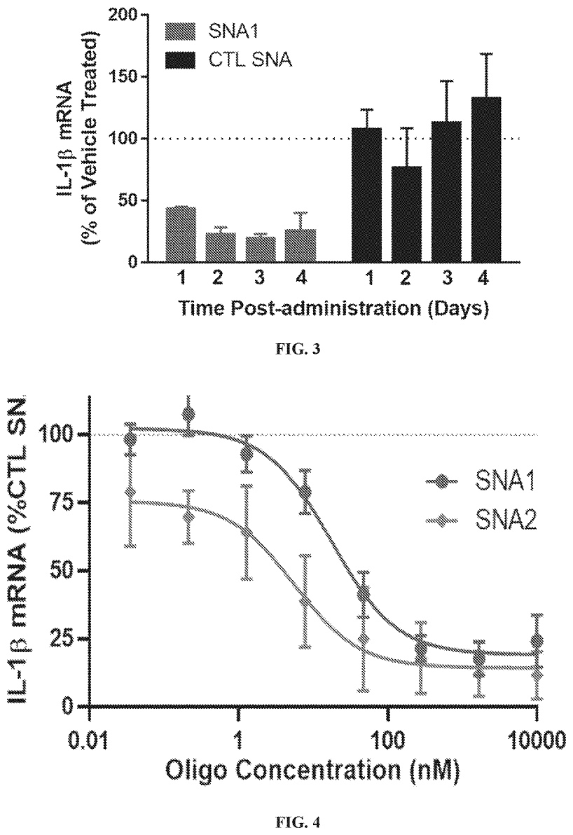

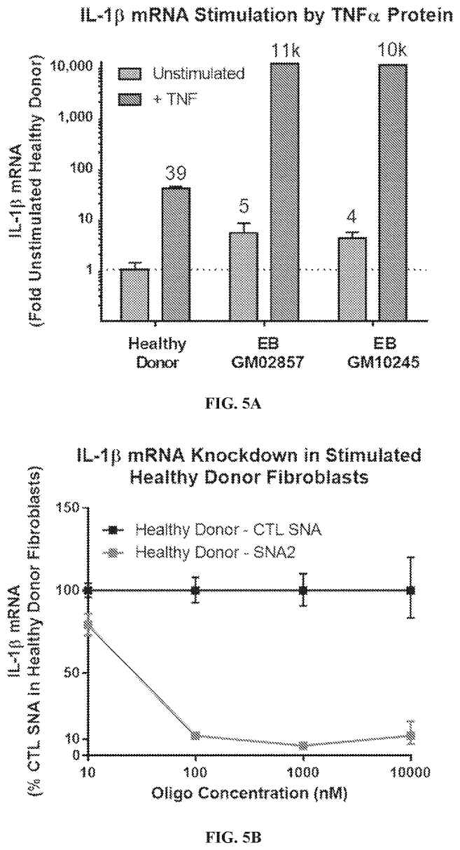

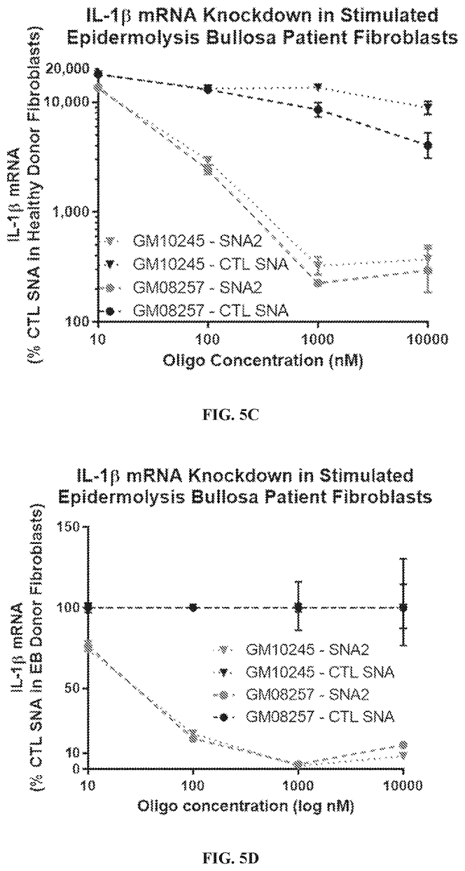

As seen in FIG. 3, after 1 day of exposure to 1000 nM SNA, IL1B mRNA knockdown persisted through at least 4 days from initiation of treatment.

### Discussion and Conclusions

After 1 day treatment with SNA, IL1B mRNA knockdown persists ≥4 days from initiation of treatment, indicating SNAs produce sustained knockdown of target mRNA.

### Example 4. Screening IL-1 Beta Targeting SNA Compounds at 1 μM Treatment Concentration

### Methods and Materials

### Cell Culture

Human neonatal foreskin keratinocytes (HFKs) were cultured in M154 media (Life Technologies) supplemented with Human Keratinocyte Growth Supplement (Life Technologies), 0.07 mM CaCl₂, 10m/mL gentamicin, and 0.25 μg/mL amphotericin B. Cells were maintained at 37° C. in a 5% CO₂ humidified incubator.

### SNA Synthesis

The SNA construct is a 3-dimensional arrangement of oligonucleotides where the nucleic acids are densely packed and radially oriented. The dense and radial arrangement of the oligonucleotides is driven by hydrophobic interactions between the cholesterol moieties at the end of the nucleic acid chains and the membrane of a liposome. The oligonucleotides and liposomes assemble spontaneously in aqueous solutions at a wide range of concentrations, thereby producing the SNA construct.

The oligonucleotides were synthesized at the 2 mole scale employing standard UniLinker support (ChemGenes). The DNA, 2′-O-Me RNA monomers and hexa(ethylene glycol) spacers were obtained from ChemGenes Corporation. The cholesterol modifier was obtained from Glen Research. Linkages were either standard phosphodiesters or phosphorothioates prepared with a solution of 0.2 M phenylacetyl disulfide (PADS) in a mixture of 1:1 lutidine:ACN. Synthesis was performed DMT-off, in the 3′ to 5′ direction. After synthesis, the oligonucleotide was cleaved from the support and de-protected using a 4:1 mixture of ammonium hydroxide and ethanol at 55° C. for 16 hours. The oligonucleotide was purified via high performance liquid chromatography (HPLC) techniques. Molecular weights and extinction coefficients were calculated using the IDT OligoAnalyzer. The verification of the oligonucleotide product molecular weights was performed using electrospray ionization mass spectrometry (ESI-MS). Finally, the oligonucleotide concentrations were determined by UV-absorbance at 260 nm on a microplate reader (BioTek).

Liposomes were formulated by first dissolving 250 mg 1,2-dioleoyl-sn-glycero-3-phosphocholine (DOPC) in chloroform to a final concentration of 50 mg/mL. The solvent was then removed under nitrogen to form a thin lipid film. The film was lyophilized overnight to ensure all solvent was removed. The lipid film was subsequently hydrated with 10 mM phosphate buffered saline (PBS) and sonication/freeze-fracture were used to form large, unilamellar vesicles. These vesicles were then continuously homogenized through a micro-fluidizer at up to 25 kpsi, until the desired mean number diameter of 20 nm was achieved. Liposomes with 50 nm diameter were synthesized by extrusion of 1,2-dioleoyl-sn-glycero-3-phosphocholine (DOPC) (NOF America) hydrated (100 g/L) in phosphate buffered saline solution (PBS) (137 mM NaCl, 10 mM phosphate, 2.7 mM KCl, pH 7.4, Hyclone) using polycarbonate membranes with 100 nm and 50 nm pores (Sterlitech). Liposome concentrations were determined using a choline quantification assay and the particle size and dispersion were measured by dynamic light scattering (DLS).

SNAs targeting human IL1B were formulated by mixing a 30 fold molar excess (20 nm liposomes) or 150 fold molar excess (50 nm liposomes) of cholesterol-modified oligonucleotides (Table 1) to the liposome suspension in PBS followed by an overnight incubation at 4° C. Similarly, a control SNA was formulated using a non-complimentary control antisense sequence (confirmed by NCBI Blast), comprising the same ‘gap-mer’ design and 3′ chemical modifications. Oligonucleotide sequences for all compounds synthesized and tested are shown in table 6.

### SNA Treatment

HFKs were seeded in 96-well, tissue culture plates at a cell density of 5,000-15,000 cells per well. Cells were allowed to rest in the incubator 1-3 days following plating until cells were ˜75% confluent and then were treated in triplicate with either an IL1B targeted antisense SNA or a non-complimentary control (confirmed by NCBI Blast) SNA, at concentrations of 10000, 1000, 100, 10, and 1 nM in fresh maintenance media.

RNA Extraction and qRT-PCR

Cells were lysed in RLT Buffer (Qiagen) at 24 hours post-transfection. RNA was isolated from lysates using the RNEasy 96-well kit (Qiagen) according to the manufacturer's instructions. cDNA was then synthesized from RNA isolates using the cDNA high capacity reverse transcription kit (Life Technologies). cDNA was prepared on a thermocycler with the following temperature program: 25° C. for 10 minutes, 37° C. for 90 minutes, 85° C. for 5 minutes followed by a 4° C. hold. The resulting cDNA was diluted 8 fold with nuclease-free water. qPCR was performed using 6 μL of the diluted cDNA, 4.66 μL LightCycler480 Probes Master Mix (Roche), 0.47 μL human IL1B specific FAM-labeled probe and primers, and 0.37 μL human Glyceraldehyde-3-phosphate dehydrogenase (GAPDH) specific HEX-labeled probe and primers per reaction well of a 384-well optical reaction plate (Roche). The primer and probe set for IL1B was purchased commercially (Hs01555410_m1, Thermo Fisher Scientific). The primer and probe set for GAPDH was designed using the known human genome sequence (NCBI reference sequences NM_002046.5 (SEQ ID NO: 145)) and was found to be specific by “blastn” analysis (NCBI). The oligonucleotide sequences used for GAPDH were: forward 5′-CAA GGT CAT CCA TGA CAA CTT TG-3′ (SEQ ID NO: 142), reverse 5′-GGG CCA TCC ACA GTC TTC T-3′ (SEQ ID NO: 143), probe 5′-HEX-ACC ACA GTC CAT GCC ATC ACT GCC A-BHQ1-3′ (SEQ ID NO: 144). qPCR reactions, in technical duplicate, were carried out on the Roche Lightcycler 480 under the following conditions: initial denaturation at 95° C. for 10 minutes and then 50 cycles of denaturation at 95° C. for 10 seconds, annealing at 60° C. for 30 seconds and extension at 72° C. for 1 second. Cp values were obtained by analysis with the 2nd derivative method. Relative gene expression was determined by normalization with the housekeeping gene (GAPDH) and the ΔΔ-Ct method.

TABLE 5
Percent Expression of IL-1 beta mRNA at 1 μM vs CTL SNA for all compounds
screened. Average of two screens and standard deviation shown where available.

| Seq | | % Average | | Liposome | Liposome: |
|---|---|---|---|---|---|
| ID | | IL1b | Standard | Diameter | Oligo |
| No: | Compound ID | Expression | Deviation | (nm) | Ratio |
| 2 | hIL1b_233 | 20 | 16 | 20 | 30 |
| 3 | hIL1b_1040 | 16 | 14 | 20 | 30 |
| 4 | hIL1b_241 | 10 | 6 | 20 | 30 |
| 5 | hIL1b_863 | 9 | 11 | 20 | 30 |
| 6 | hIL1b_178 | 12 | 11 | 20 | 30 |
| 7 | hIL1b_599 | 18 | 19 | 20 | 30 |
| 8 | hIL1b_1170 | 20 | 19 | 20 | 30 |
| 9 | hIL1b_g8196 | 14 | 9 | 20 | 30 |
| 10 | hIL1b_750 | 16 | 6 | 20 | 30 |
| 11 | hIL1b_864 | 16 | 12 | 20 | 30 |
| 12 | hIL1b_397 | 18 | 17 | 20 | 30 |
| 13 | hIL1b_235 | 12 | 8 | 20 | 30 |
| 14 | hIL1b_242 | 20 | 17 | 20 | 30 |
| 15 | hIL1b_277 | 12 | 13 | 20 | 30 |
| 16 | hIL1b_1409 | 21 | 16 | 20 | 30 |
| 17 | hIL1b_805 | 18 | 10 | 20 | 30 |
| 18 | hIL1b_748 | 21 | 16 | 20 | 30 |
| 19 | hIL1b_g8398 | 71 | 41 | 20 | 30 |
| 20 | hIL1b_473 | 28 | 17 | 20 | 30 |
| 21 | hIL1b_1412 | 43 | 43 | 20 | 30 |
| 22 | hIL1b_1145-5MdzG | 33 | 26 | 20 | 30 |
| 23 | hIL1b_g5527 | 68 | 46 | 20 | 30 |
| 24 | hIL1b_1211-14MdzG | 44 | 35 | 20 | 30 |
| 25 | hIL1b_g5589 | 56 | 16 | 20 | 30 |
| 26 | hIL1b_1231-9dzG | 48 | 50 | 20 | 30 |
| 27 | hIL1b_813-4MdzG | 52 | 45 | 20 | 30 |
| 28 | hIL1b_693-5,16MdzG | 39 | 39 | 20 | 30 |
| 29 | hIL1b_126 | 53 | 57 | 20 | 30 |
| 30 | hIL1b_698-10dzG | 71 | 63 | 20 | 30 |
| 31 | hIL1b_g8945 | 123 | | 20 | 30 |
| 32 | hIL1b_1208-7dzG | 52 | 33 | 20 | 30 |
| 33 | hIL1b_545 | 59 | 39 | 20 | 30 |
| 34 | hIL1b_1229-7dzG | 42 | 28 | 20 | 30 |
| 35 | hIL1b_1207-10dzG | 50 | 35 | 20 | 30 |
| 36 | hIL1b_1186-5MdzG | 54 | 45 | 20 | 30 |
| 37 | hIL1b_1230-8dzG | 44 | 48 | 20 | 30 |
| 38 | hIL1b_1185-4MdzG | 65 | 0 | 20 | 30 |
| 39 | hIL1b_g6154 | 56 | 34 | 20 | 30 |
| 40 | hIL1b_g10480 | 73 | 13 | 20 | 30 |
| 41 | hIL1b_g11198 | 76 | 18 | 20 | 30 |
| 42 | hIL1b_676 | 37 | 21 | 20 | 30 |
| 43 | hIL1b_241-12m5C | 20 | 1 | 20 | 30 |
| 44 | hIL1b_389 | 155 | 132 | 50 | 150 |
| 45 | hIL1b_390 | 162 | 148 | 50 | 150 |
| 46 | hIL1b_399 | 41 | | 50 | 150 |
| 47 | hIL1b_400 | 47 | | 50 | 150 |
| 48 | hIL1b_401 | 31 | | 50 | 150 |
| 49 | hIL1b_402 | 37 | | 50 | 150 |
| 50 | hIL1b_403 | 103 | 63 | 50 | 150 |
| 51 | hIL1b_404 | 126 | 111 | 50 | 150 |
| 52 | ENA_hIL1b_405-Chol | 47 | | 50 | 150 |
| 53 | hIL1b_405 | 29 | 8 | 50 | 150 |
| 54 | hIL1b_405(3/12/3) | 20 | | 50 | 150 |
| 55 | hIL1b_405(5/6/7) | 28 | | 50 | 150 |
| 56 | hIL1b_405(5/8/5) | 21 | | 50 | 150 |
| 57 | hIL1b_405(7/6/5) | 22 | | 50 | 150 |
| 58 | hIL1b_405(9/6/3) | 14 | | 50 | 150 |
| 59 | hIL1b_405-NoLinker | 63 | 10 | 50 | 150 |
| 60 | hIL1b_405-PEG2500 | 71 | 22 | 50 | 150 |
| 61 | hIL1b_406 | 33 | | 50 | 150 |
| 62 | hIL1b_407 | 29 | | 50 | 150 |
| 63 | hIL1b_408 | 35 | | 50 | 150 |
| 64 | hIL1b_409 | 46 | | 50 | 150 |
| 65 | hIL1b_507 | 24 | | 50 | 150 |
| 66 | hIL1b_508 | 31 | | 50 | 150 |
| 67 | hIL1b_509 | 29 | | 50 | 150 |
| 68 | hIL1b_510 | 41 | | 50 | 150 |
| 69 | hIL1b_511 | 39 | | 50 | 150 |
| 70 | hIL1b_512 | 33 | | 50 | 150 |
| 71 | hIL1b_513 | 46 | | 50 | 150 |
| 72 | hIL1b_514 | 38 | | 50 | 150 |
| 73 | hIL1b_515 | 101 | | 50 | 150 |
| 74 | hIL1b_516 | 28 | 2 | 50 | 150 |
| 75 | hIL1b_516(3/12/3) | 27 | | 50 | 150 |
| 76 | hIL1b_516(3/6/9) | 23 | | 50 | 150 |
| 77 | hIL1b_516(5/6/7) | 26 | | 50 | 150 |
| 78 | hIL1b_516(5/8/5) | 71 | 21 | 50 | 150 |
| 79 | hIL1b_516(7/6/5) | 38 | | 50 | 150 |
| 80 | hIL1b_516(9/6/3) | 60 | | 50 | 150 |
| 81 | hIL1b_517 | 50 | | 50 | 150 |
| 82 | hIL1b_587 | 94 | | 50 | 150 |
| 83 | hIL1b_588 | 169 | 177 | 50 | 150 |
| 84 | hIL1b_590 | 128 | 119 | 50 | 150 |
| 85 | hIL1b_591 | 99 | | 50 | 150 |
| 86 | hIL1b_592 | 99 | 95 | 50 | 150 |
| 87 | hIL1b_593 | 96 | 81 | 50 | 150 |
| 88 | hIL1b_594 | 44 | | 50 | 150 |
| 89 | hIL1b_595 | 223 | 250 | 50 | 150 |
| 90 | hIL1b_596 | 247 | 300 | 50 | 150 |
| 91 | hIL1b_597 | 79 | | 50 | 150 |
| 92 | hIL1b_598 | 273 | 307 | 50 | 150 |
| 93 | hIL1b_600 | 154 | 119 | 50 | 150 |
| 94 | hIL1b_696 | 128 | 65 | 50 | 150 |
| 95 | hIL1b_697 | 129 | 60 | 50 | 150 |
| 96 | hIL1b_698 | 137 | 51 | 50 | 150 |
| 97 | hIL1b_699 | 134 | 50 | 50 | 150 |
| 98 | hIL1b_751 | 82 | 31 | 50 | 150 |
| 99 | hIL1b_753 | 84 | 6 | 50 | 150 |
| 100 | hIL1b_755 | 116 | 34 | 50 | 150 |
| 101 | hIL1b_781 | 110 | 59 | 50 | 150 |
| 102 | hIL1b_783 | 93 | 47 | 50 | 150 |
| 103 | hIL1b_785 | 58 | 18 | 50 | 150 |
| 104 | hIL1b_791 | 49 | | 50 | 150 |
| 105 | hIL1b_792 | 37 | | 50 | 150 |
| 106 | hIL1b_793 | 116 | 58 | 50 | 150 |
| 107 | hIL1b_794 | 79 | 4 | 50 | 150 |
| 108 | hIL1b_795 | 24 | | 50 | 150 |
| 109 | hIL1b_796 | 77 | 21 | 50 | 150 |
| 110 | hIL1b_797 | 98 | 31 | 50 | 150 |
| 111 | hIL1b_798 | 37 | | 50 | 150 |
| 112 | hIL1b_799 | 38 | | 50 | 150 |
| 113 | hIL1b_800 | 37 | | 50 | 150 |
| 114 | hIL1b_801 | 38 | | 50 | 150 |
| 115 | hIL1b_812 | 118 | 55 | 50 | 150 |
| 116 | hIL1b_814 | 148 | 70 | 50 | 150 |
| 117 | hIL1b_816 | 131 | 87 | 50 | 150 |
| 118 | hIL1b_817 | 181 | 129 | 50 | 150 |
| 119 | hIL1b_867 | 145 | 118 | 50 | 150 |
| 120 | hIL1b_868 | 131 | 97 | 50 | 150 |
| 121 | hIL1b_869 | 146 | 114 | 50 | 150 |
| 122 | hIL1b_870 | 112 | 62 | 50 | 150 |
| 123 | hIL1b_871 | 145 | 102 | 50 | 150 |
| 124 | hIL1b_1008 | 110 | 87 | 50 | 150 |
| 125 | hIL1b_1009 | 127 | | 50 | 150 |
| 126 | hIL1b_1010 | 105 | 91 | 50 | 150 |
| 127 | hIL1b_1011 | 31 | | 50 | 150 |
| 128 | hIL1b_1012 | 103 | 83 | 50 | 150 |
| 129 | hIL1b_1013 | 32 | | 50 | 150 |
| 130 | hIL1b_1014 | 40 | | 50 | 150 |
| 131 | hIL1b_1015 | 42 | | 50 | 150 |
| 132 | hIL1b_1187 | 194 | 189 | 50 | 150 |
| 133 | hIL1b_1189 | 97 | 82 | 50 | 150 |
| 134 | hIL1b_1192 | 103 | 56 | 50 | 150 |
| 135 | hIL1b_1232 | 76 | 49 | 50 | 150 |
| 136 | hIL1b_1234 | 107 | 49 | 50 | 150 |
| 137 | hIL1b_1236 | 119 | 113 | 50 | 150 |
| 138 | hIL1b_1404 | 113 | 67 | 50 | 150 |
| 139 | hIL1b_1406 | 359 | 423 | 50 | 150 |
| 140 | hIL1B_241 | 18 | 0 | 20 | 30 |
| 141 | hIL1b_241(5/6/7) | 8 | 1 | 20 | 30 |

### Example 5. Dose Response Curve of Selected SNA Compounds

Dose response of selected SNA compounds was determined in HFK cells. Cells were treated as described above and IL1B mRNA levels were measured by qRT-PCR as described above. Two tested compounds showed excellent dose response with IC₅₀ of ˜17.5 nM for SNA1 and 5.5 nM for SNA2 (FIG. 4).

### Example 6. SNAs Normalize the Dysregulated IL-1β Expression in Epidermolysis Bullosa Patient Cells

TABLE 6
All anti-IL1B sequences synthesized and tested as SNA.

| Seq ID | | |
|---|---|---|
| No: | Compound ID | Compound Sequence |
| 2 | hIL1b_233 | mGmGmUmCmGmGA*G*A*T*T*C*mGmUmAmGmCmU/isp18//isp18//3CholTEG/ |
| 3 | hIL1b_1040 | mAmGmAmUmCmCT*C*T*T*A*G*mCmAmCmUmAmC/isp18//isp18//3CholTEG/ |
| 4 | hIL1b_241 | mGmUmAmGmUmGG*T*G*G*T*C*mGmGmAmGmAmU/isp18//isp18//3CholTEG/ |
| 5 | hIL1b_863 | mGmCmAmUmGmGT*G*A*A*G*T*mCmAmGmUmUmA/isp18//isp18//3CholTEG/ |
| 6 | hIL1b_178 | mGmAmAmGmGmAG*C*A*C*T*T*mCmAmUmCmUmG/isp18//isp18//3CholTEG/ |
| 7 | hIL1b_599 | mCmCmAmAmGmGC*C*A*C*A*G*mGmUmAmUmUmU/isp18//isp18//3CholTEG/ |
| 8 | hIL1b_1170 | mCmCmAmAmAmUG*T*G*G*C*C*mGmUmGmGmUmU/isp18//isp18//3CholTEG/ |
| 9 | hIL1b_g8196 | mGmAmAmGmGmAG*C*A*C*T*G*mCmGmGmAmGmA/isp18//isp18//3CholTEG/ |
| 10 | hIL1b_750 | mUmCmCmAmGmCT*T*G*T*T*A*mUmUmGmAmUmU/isp18//isp18//3CholTEG/ |
| 11 | hIL1b_864 | mUmGmCmAmUmGG*T*G*A*A*G*mUmCmAmGmUmU/isp18//isp18//3CholTEG/ |
| 12 | hIL1b_397 | mAmUmCmCmCmAT*G*T*G*T*C*mGmAmAmGmAmA/isp18//isp18//3CholTEG/ |
| 13 | hIL1b_235 | mGmUmGmGmUmCG*G*A*G*A*T*mUmCmGmUmAmG/isp18//isp18//3CholTEG/ |
| 14 | hIL1b_242 | mUmGmUmAmGmUG*G*T*G*G*T*mCmGmGmAmGmA/isp18//isp18//3CholTEG/ |
| 15 | hIL1b_277 | mCmAmCmAmAmCA*A*C*T*G*A*mCmGmCmGmGmC/isp18//isp18//3CholTEG/ |
| 16 | hIL1b_1409 | mAmAmUmCmUmGA*G*C*T*T*A*mUmAmUmAmUmU/isp18//isp18//3CholTEG/ |
| 17 | hIL1b_805 | mGmUmUmUmUmCT*G*C*T*T*G*mAmGmAmGmGmU/isp18//isp18//3CholTEG/ |
| 18 | hIL1b_748 | mCmAmGmCmUmUG*T*T*A*T*T*mGmAmUmUmUmC/isp18//isp18//3CholTEG/ |
| 19 | hIL1b_g8398 | mGmCmUmAmAmCT*A*C*C*T*T*mCmUmUmCmAmA/isp18//isp18//3CholTEG/ |
| 20 | hIL1b_473 | mCmCmAmAmGmCT*T*T*T*T*T*mGmCmUmGmUmG/isp18//isp18//3CholTEG/ |
| 21 | hIL1b_1412 | mAmAmUmAmAmUC*T*G*A*G*C*mUmUmAmUmAmU/isp18//isp18//3CholTEG/ |
| 22 | hIL1b_1145-5MdzG | mGmGmCmG/7deAzaOMG/mGC*T*T*T*A*A*mGmUmGmAmGmU/isp18//isp18//3Chol |
| | | TEG/ |
| 23 | hIL1b_g5527 | mUmUmCmAmGmAC*A*C*C*T*G*mUmGmUmAmAmA/isp18//isp18//3CholTEG/ |
| 24 | hIL1b_1211-14MdzG | mUmCmAmUmCmAG*A*A*T*G*T*mG/7deAzaOMG/mGmAmGmC/isp18//isp18//3Chol |
| | | TEG/ |
| 25 | hIL1b_g5589 | mUmCmCmAmCmUG*A*C*C*T*G*mUmAmAmUmAmA/isp18//isp18//3CholTEG/ |
| 26 | hIL1b_1231-9dzG | mAmUmAmAmAmUA*G*/7deAzaG/*G*A*A*mGmCmGmGmUmU/isp18//isp18//3CholT |
| | | EG/ |
| 27 | hIL1b_813-4MdzG | mAmCmG/7deAzaOMG/mGmCA*T*G*T*T*T*mUmCmUmGmCmU/isp18//isp18//3Chol |
| | | TEG/ |
| 28 | hIL1b_693- | mUmUmUmG/7deAzaOMG/mGT*A*A*T*T*T*mUmUmG/7deAzaOMG/mGmA/isp18//is |
| | 5, 16MdzG | p18//3CholTEG/ |
| 29 | hIL1b_126 | mUmCmAmUmUmGC*C*A*C*T*G*mUmAmAmUmAmA/isp18//isp18//3CholTEG/ |
| 30 | hIL1b_698-10dzG | mUmCmUmUmCmUT*T*G*/7deAzaG/*G*T*mAmAmUmUmUmU/isp18//isp18//3CholT |
| | | EG/ |
| 31 | hIL1b_g8945 | mAmGmAmUmAmGG*T*T*C*T*G*mAmAmAmUmGmU/isp18//isp18//3CholTEG/ |
| 32 | hIL1b_1208-7dzG | mUmCmAmGmAmAT*G*T*G*/7deAzaG/*G*mAmGmCmGmAmA/isp18//isp18//3CholT |
| | | EG/ |
| 33 | hIL1b_545 | mAmGmAmAmCmAC*C*A*C*T*T*mGmUmUmGmCmU/isp18//isp18//3CholTEG/ |
| 34 | hIL1b_1229-7dzG | mAmAmAmUmAmG/7deAzaG/*G*A*A*G*C*mGmGmUmUmGmC/isp18//isp18//3CholT |
| | | EG/ |
| 35 | hIL1b_1207-10dzG | mCmAmGmAmAmUG*T*G*/7deAzaG/*G*A*mGmCmGmAmAmU/isp18//isp18//3CholT |
| | | EG/ |
| 36 | hIL1b_1186-5MdzG | mAmGmAmG/7deAzaOMG/mGT*T*T*C*T*T*mAmGmAmAmCmC/isp18//isp18//3Chol |
| | | TEG/ |
| 37 | hIL1b_1230-8dzG | mUmAmAmAmUmAG*/7deAzaG/*G*A*A*G*mCmGmGmUmUmG/isp18//isp18//3CholT |
| | | EG/ |
| 38 | hIL1b_1185-4MdzG | mGmAmG/7deAzaOMG/mGmUT*T*C*T*T*A*mGmAmAmCmCmA/isp18//isp18//3Chol |
| | | TEG/ |
| 39 | hIL1b_g6154 | mUmCmAmUmUmGC*C*A*C*T*G*mUmAmAmUmAmA/isp18//isp18//3CholTEG/ |
| 40 | hIL1b_g10480 | mUmUmCmAmCmUT*A*C*C*T*C*mCmAmGmCmUmG/isp18//isp18//3CholTEG/ |
| 41 | hIL1b_g11198 | mAmUmCmUmAmCA*C*T*C*T*G*mCmAmGmCmGmA/isp18//isp18//3CholTEG/ |
| 42 | hIL1b_676 | mAmUmCmUmAmCA*C*T*C*T*C*mCmAmGmCmUmG/isp18//isp18//3CholTEG/ |
| 43 | hIL1b_241-12m5C | mGmUmAmGmUmGG*T*G*G*T*/iMe- |
| | | dC/*mGmGmAmGmAmU/isp18//isp18//3CholTEG/ |
| 44 | hIL1b_389 | mUmGmUmCmGmAA*G*A*A*G*A*mUmAmGmGmUmU/isp18//isp18//3CholTEG/ |
| 45 | hIL1b_390 | mGmUmGmUmCmGA*A*G*A*A*G*mAmUmAmGmGmU/isp18//isp18//3CholTEG/ |
| 46 | hIL1b_399 | mUmUmAmUmCmCC*A*T*G*T*G*mUmCmGmAmAmG/isp18//isp18//3CholTEG/ |
| 47 | hIL1b_400 | mGmUmUmAmUmCC*C*A*T*G*T*mGmUmCmGmAmA/isp18//isp18//3CholTEG/ |
| 48 | hIL1b_401 | mCmGmUmUmAmUC*C*C*A*T*G*mUmGmUmCmGmA/isp18//isp18//3CholTEG/ |
| 49 | hIL1b_402 | mUmCmGmUmUmAT*C*C*C*A*T*mGmUmGmUmCmG/isp18//isp18//3CholTEG/ |
| 50 | hIL1b_403 | mCmUmCmGmUmUA*T*C*C*C*A*mUmGmUmGmUmC/isp18//isp18//3CholTEG/ |
| 51 | hIL1b_404 | mCmCmUmCmGmUT*A*T*C*C*C*mAmUmGmUmGmU/isp18//isp18//3CholTEG/ |
| 52 | ENA_hIL1b_405- | eGeCeCeUeCeGT*T*A*T*C*C*eCeAeUeGeUeG/isp18//isp18//3CholTEG/ |
| | Chol | |
| 53 | hIL1b_405 | mGmCmCmUmCmGT*T*A*T*C*C*mCmAmUmGmUmG/isp18//isp18//3CholTEG/ |
| 54 | hIL1b_405(3/12/3) | mGmCmCT*C*G*T*T*A*T*C*C*C*A*T*mGmUmG/isp18//isp18//3CholTEG/ |
| 55 | hIL1b_405(5/6/7) | mGmCmCmUmCG*T*T*A*T*C*mCmCmAmUmGmUmG/isp18//isp18//3CholTEG/ |
| 56 | hIL1b_405(5/8/5) | mGmCmCmUmCG*T*T*A*T*C*C*C*mAmUmGmUmG/isp18//isp18//3CholTEG/ |
| 57 | hIL1b_405(7/6/5) | mGmCmCmUmCmGmUT*A*T*C*C*C*mAmUmGmUmG/isp18//isp18//3CholTEG/ |
| 58 | hIL1b_405(9/6/3) | mGmCmCmUmCmGmUmUmAT*C*C*C*A*T*mGmUmG/isp18//isp18//3CholTEG/ |
| 59 | hIL1b_405-NoLinker | mGmCmCmUmCmGT*T*A*T*C*C*mCmAmUmGmUmG/3CholTEG/ |
| 60 | hIL1b_405-PEG2500 | mGmCmCmUmCmGT*T*A*T*C*C*mCmAmUmGmUmG//PEG2500/3CholTEG/ |
| 61 | hIL1b_406 | mAmGmCmCmUmCG*T*T*A*T*C*mCmCmAmUmGmU/isp18//isp18//3CholTEG/ |
| 62 | hIL1b_407 | mAmAmGmCmCmUC*G*T*T*A*T*mCmCmCmAmUmG/isp18//isp18//3CholTEG/ |
| 63 | hIL1b_408 | mUmAmAmGmCmCT*C*G*T*T*A*mUmCmCmCmAmU/isp18//isp18//3CholTEG/ |
| 64 | hIL1b_409 | mAmUmAmAmGmCC*T*C*G*T*T*mAmUmCmCmCmA/isp18//isp18//3CholTEG/ |
| 65 | hIL1b_507 | mUmGmGmAmGmAG*C*T*T*T*C*mAmGmUmUmCmA/isp18//isp18//3CholTEG/ |
| 66 | hIL1b_508 | mGmUmGmGmAmGA*G*C*T*T*T*mCmAmGmUmUmC/isp18//isp18//3CholTEG/ |
| 67 | hIL1b_509 | mGmGmUmGmGmAG*A*G*C*T*T*mUmCmAmGmUmU/isp18//isp18//3CholTEG/ |
| 68 | hIL1b_510 | mAmGmGmUmGmGA*G*A*G*C*T*mUmUmCmAmGmU/isp18//isp18//3CholTEG/ |
| 69 | hIL1b_511 | mGmAmGmGmUmGG*A*G*A*G*C*mUmUmUmCmAmG/isp18//isp18//3CholTEG/ |
| 70 | hIL1b_512 | mGmGmAmGmGmUG*G*A*G*A*G*mCmUmUmUmCmA/isp18//isp18//3CholTEG/ |
| 71 | hIL1b_513 | mUmGmGmAmGmGT*G*G*A*G*A*mGmCmUmUmUmC/isp18//isp18//3CholTEG/ |
| 72 | hIL1b_514 | mCmUmGmGmAmGG*T*G*G*A*G*mAmGmCmUmUmU/isp18//isp18//3CholTEG/ |
| 73 | hIL1b_515 | mCmCmUmGmGmAG*G*T*G*G*A*mGmAmGmCmUmU/isp18//isp18//3CholTEG/ |
| 74 | hIL1b_516 | mCmCmCmUmGmGA*G*G*T*G*G*mAmGmAmGmCmU/isp18//isp18//3CholTEG/ |
| 75 | hIL1b_516(3/12/3) | mCmCmCT*G*G*A*G*G*T*G*G*A*G*A*mGmCmU/isp18//isp18//3CholTEG/ |
| 76 | hIL1b_516(3/6/9) | mCmCmCT*G*G*A*G*G*mUmGmGmAmGmAmGmCmU/isp18//isp18//3CholTEG/ |
| 77 | hIL1b_516(5/6/7) | mCmCmCmUmGG*A*G*G*T*G*mGmAmGmAmGmCmU/isp18//isp18//3CholTEG/ |
| 78 | hIL1b_516(5/8/5) | mCmCmCmUmGG*A*G*G*T*G*G*A*mGmAmGmCmU/isp18//isp18//3CholTEG/ |
| 79 | hIL1b_516(7/6/5) | mCmCmCmUmGmGmAG*G*T*G*G*A*mGmAmGmCmU/isp18//isp18//3CholTEG/ |
| 80 | hIL1b_516(9/6/3) | mCmCmCmUmGmGmAmGmGT*G*G*A*G*A*mGmCmU/isp18//isp18//3CholTEG/ |
| 81 | hIL1b_517 | mUmCmCmCmUmGG*A*G*G*T*G*mGmAmGmAmGmC/isp18//isp18//3CholTEG/ |
| 82 | hIL1b_587 | mGmUmAmUmUmUT*G*T*C*A*T*mUmAmCmUmUmU/isp18//isp18//3CholTEG/ |
| 83 | hIL1b_588 | mGmGmUmAmUmUT*T*G*T*C*A*mUmUmAmCmUmU/isp18//isp18//3CholTEG/ |
| 84 | hIL1b_590 | mCmAmGmGmUmAT*T*T*T*G*T*mCmAmUmUmAmC/isp18//isp18//3CholTEG/ |
| 85 | hIL1b_591 | mAmCmAmGmGmUA*T*T*T*T*G*mUmCmAmUmUmA/isp18//isp18//3CholTEG/ |
| 86 | hIL1b_592 | mCmAmCmAmGmGT*A*T*T*T*T*mGmUmCmAmUmU/isp18//isp18//3CholTEG/ |
| 87 | hIL1b_593 | mCmCmAmCmAmGG*T*A*T*T*T*mUmGmUmCmAmU/isp18//isp18//3CholTEG/ |
| 88 | hIL1b_594 | mGmCmCmAmCmAG*G*T*A*T*T*mUmUmGmUmCmA/isp18//isp18//3CholTEG/ |
| 89 | hIL1b_595 | mGmGmCmCmAmCA*G*G*T*A*T*mUmUmUmGmUmC/isp18//isp18//3CholTEG/ |
| 90 | hIL1b_596 | mAmGmGmCmCmAC*A*G*G*T*A*mUmUmUmUmGmU/isp18//isp18//3CholTEG/ |
| 91 | hIL1b_597 | mAmAmGmGmCmCA*C*A*G*G*T*mAmUmUmUmUmG/isp18//isp18//3CholTEG/ |
| 92 | hIL1b_598 | mCmAmAmGmGmCC*A*C*A*G*G*mUmAmUmUmUmU/isp18//isp18//3CholTEG/ |
| 93 | hIL1b_600 | mCmCmCmAmAmGG*C*C*A*C*A*mGmGmUmAmUmU/isp18//isp18//3CholTEG/ |
| 94 | hIL1b_696 | mUmUmCmUmUmUG*G*G*T*A*A*mUmUmUmUmUmG/isp18//isp18//3CholTEG/ |
| 95 | hIL1b_697 | mCmUmUmCmUmUT*G*G*G*T*A*mAmUmUmUmUmU/isp18//isp18//3CholTEG/ |
| 96 | hIL1b_698 | mUmCmUmUmCmUT*T*G*G*G*T*mAmAmUmUmUmU/isp18//isp18//3CholTEG/ |
| 97 | hIL1b_699 | mUmUmCmUmUmCT*T*T*G*G*G*mUmAmAmUmUmU/isp18//isp18//3CholTEG/ |
| 98 | hIL1b_751 | mUmUmCmCmAmGC*T*T*G*T*T*mAmUmUmGmAmU/isp18//isp18//3CholTEG/ |
| 99 | hIL1b_753 | mAmAmUmUmCmCA*G*C*T*T*G*mUmUmAmUmUmG/isp18//isp18//3CholTEG/ |
| 100 | hIL1b_755 | mCmAmAmAmUmUC*C*A*G*C*T*mUmGmUmUmAmU/isp18//isp18//3CholTEG/ |
| 101 | hIL1b_781 | mGmUmAmCmCmAG*T*T*G*G*G*mGmAmAmCmUmG/isp18//isp18//3CholTEG/ |
| 102 | hIL1b_783 | mAmUmGmUmAmCC*A*G*T*T*G*mGmGmGmAmAmC/isp18//isp18//3CholTEG/ |
| 103 | hIL1b_785 | mUmGmAmUmGmUA*C*C*A*G*T*mUmGmGmGmGmA/isp18//isp18//3CholTEG/ |
| 104 | hIL1b_791 | mAmGmGmUmGmCT*G*A*T*G*T*mAmCmCmAmGmU/isp18//isp18//3CholTEG/ |
| 105 | hIL1b_792 | mGmAmGmGmUmGC*T*G*A*T*G*mUmAmCmCmAmG/isp18//isp18//3CholTEG/ |
| 106 | hIL1b_793 | mAmGmAmGmGmUG*C*T*G*A*T*mGmUmAmCmCmA/isp18//isp18//3CholTEG/ |
| 107 | hIL1b_794 | mGmAmGmAmGmGT*G*C*T*G*A*mUmGmUmAmCmC/isp18//isp18//3CholTEG/ |
| 108 | hIL1b_795 | mUmGmAmGmAmGG*T*G*C*T*G*mAmUmGmUmAmC/isp18//isp18//3CholTEG/ |
| 109 | hIL1b_796 | mUmUmGmAmGmAG*G*T*G*C*T*mGmAmUmGmUmA/isp18//isp18//3CholTEG/ |
| 110 | hIL1b_797 | mCmUmUmGmAmGA*G*G*T*G*C*mUmGmAmUmGmU/isp18//isp18//3CholTEG/ |
| 111 | hIL1b_798 | mGmCmUmUmGmAG*A*G*G*T*G*mCmUmGmAmUmG/isp18//isp18//3CholTEG/ |
| 112 | hIL1b_799 | mUmGmCmUmUmGA*G*A*G*G*T*mGmCmUmGmAmU/isp18//isp18//3CholTEG/ |
| 113 | hIL1b_800 | mCmUmGmCmUmUG*A*G*A*G*G*mUmGmCmUmGmA/isp18//isp18//3CholTEG/ |
| 114 | hIL1b_801 | mUmCmUmGmCmUT*G*A*G*A*G*mGmUmGmCmUmG/isp18//isp18//3CholTEG/ |
| 115 | hIL1b_812 | mCmGmGmGmCmAT*G*T*T*T*T*mCmUmGmCmUmU/isp18//isp18//3CholTEG/ |
| 116 | hIL1b_814 | mGmAmCmGmGmGC*A*T*G*T*T*mUmUmCmUmGmC/isp18//isp18//3CholTEG/ |
| 117 | hIL1b_816 | mAmAmGmAmCmGG*G*C*A*T*G*mUmUmUmUmCmU/isp18//isp18//3CholTEG/ |
| 118 | hIL1b_817 | mGmAmAmGmAmCG*G*G*C*A*T*mGmUmUmUmUmC/isp18//isp18//3CholTEG/ |
| 119 | hIL1b_867 | mAmAmUmUmGmCA*T*G*G*T*G*mAmAmGmUmCmA/isp18//isp18//3CholTEG/ |
| 120 | hIL1b_868 | mAmAmAmUmUmGC*A*T*G*G*T*mGmAmAmGmUmC/isp18//isp18//3CholTEG/ |
| 121 | hIL1b_869 | mCmAmAmAmUmUG*C*A*T*G*G*mUmGmAmAmGmU/isp18//isp18//3CholTEG/ |
| 122 | hIL1b_870 | mAmCmAmAmAmUT*G*C*A*T*G*mGmUmGmAmAmG/isp18//isp18//3CholTEG/ |
| 123 | hIL1b_871 | mCmAmCmAmAmAT*T*G*C*A*T*mGmGmUmGmAmA/isp18//isp18//3CholTEG/ |
| 124 | hIL1b_1008 | mUmUmGmGmGmCA*T*T*G*G*T*mGmUmAmGmAmC/isp18//isp18//3CholTEG/ |
| 125 | hIL1b_1009 | mGmUmUmGmGmGC*A*T*T*G*G*mUmGmUmAmGmA/isp18//isp18//3CholTEG/ |
| 126 | hIL1b_1010 | mAmGmUmUmGmGG*C*A*T*T*G*mGmUmGmUmAmG/isp18//isp18//3CholTEG/ |
| 127 | hIL1b_1011 | mCmAmGmUmUmGG*G*C*A*T*T*mGmGmUmGmUmA/isp18//isp18//3CholTEG/ |
| 128 | hIL1b_1012 | mGmCmAmGmUmUG*G*G*C*A*T*mUmGmGmUmGmU/isp18//isp18//3CholTEG/ |
| 129 | hIL1b_1013 | mGmGmCmAmGmUT*G*G*G*C*A*mUmUmGmGmUmG/isp18//isp18//3CholTEG/ |
| 130 | hIL1b_1014 | mAmGmGmCmAmGT*T*G*G*G*C*mAmUmUmGmGmU/isp18//isp18//3CholTEG/ |
| 131 | hIL1b_1015 | mCmAmGmGmCmAG*T*T*G*G*G*mCmAmUmUmGmG/isp18//isp18//3CholTEG/ |
| 132 | hIL1b_1187 | mCmAmGmAmGmGG*T*T*T*C*T*mUmAmGmAmAmC/isp18//isp18//3CholTEG/ |
| 133 | hIL1b_1189 | mGmAmCmAmGmAG*G*G*T*T*T*mCmUmUmAmGmA/isp18//isp18//3CholTEG/ |
| 134 | hIL1b_1192 | mAmAmUmGmAmCA*G*A*G*G*G*mUmUmUmCmUmU/isp18//isp18//3CholTEG/ |
| 135 | hIL1b_1232 | mAmAmUmAmAmAT*A*G*G*G*A*mAmGmCmGmGmU/isp18//isp18//3CholTEG/ |
| 136 | hIL1b_1234 | mUmAmAmAmUmAA*A*T*A*G*G*mGmAmAmGmCmG/isp18//isp18//3CholTEG/ |
| 137 | hIL1b_1236 | mAmAmUmAmAmAT*A*A*A*T*A*mGmGmGmAmAmG/isp18//isp18//3CholTEG/ |
| 138 | hIL1b_1404 | mGmAmGmCmUmUA*T*A*T*A*T*mUmUmUmCmAmG/isp18//isp18//3CholTEG/ |
| 139 | hIL1b_1406 | mCmUmGmAmGmCT*T*A*T*A*T*mAmUmUmUmUmC/isp18//isp18//3CholTEG/ |
| 140 | hIL1B_241 | mGmUmAmGmUmGG*T*G*G*T*C*mGmGmAmGmAmU/isp18//isp18//3CholTEG/ |
| 141 | hIL1b_241(5/6/7) | mGmUmAmGmUG*G*T*G*G*T*mCmGmGmAmGmAmU/isp18//isp18//3CholTEG/ |

“*” denotes a phosphorothioate bond, “m” denotes an 2′-O-methylated base, “/iSp18/” denotes a hexa(ethylene glycol) spacer, “/3CholTEG/” denotes a 3′ tri(ethylene glycol) bound to a cholesteryl ester, 5-Me-C denotes 5 methyl cytosine, PEG2500 denotes polyethylene glycol average molecular weight 2,500 dalton, 7deAzaG denotes 7-deaza-deoxyguanosine, 7deAzaOMG denotes 2′-O-methyl 7-deaza-deoxyguanosine.

Human IL-1 beta (NM_000576)
SEQ ID NO: 1
accaaacctcttcgaggcacaaggcacaacaggctgctctgggattctctt
cagccaatcttcattgctcaagtgtctgaagcagccatggcagaagtacct
gagctcgccagtgaaatgatggcttattacagtggcaatgaggatgacttg
ttctttgaagctgatggccctaaacagatgaagtgctccttccaggacctg
gacctctgccctctggatggcggcatccagctacgaatctccgaccaccac
tacagcaagggcttcaggcaggccgcgtcagttgttgtggccatggacaag
ctgaggaagatgctggttccctgcccacagaccttccaggagaatgacctg
agcaccttctttcccttcatctttgaagaagaacctatcttcttcgacaca
tgggataacgaggcttatgtgcacgatgcacctgtacgatcactgaactgc
acgctccgggactcacagcaaaaaagcttggtgatgtctggtccatatgaa
ctgaaagctctccacctccagggacaggatatggagcaacaagtggtgttc
tccatgtcctttgtacaaggagaagaaagtaatgacaaaatacctgtggcc
ttgggcctcaaggaaaagaatctgtacctgtcctgcgtgttgaaagatgat
aagcccactctacagctggagagtgtagatcccaaaaattacccaaagaag
aagatggaaaagcgatttgtcttcaacaagatagaaatcaataacaagctg
gaatttgagtctgcccagttccccaactggtacatcagcacctctcaagca
gaaaacatgcccgtcttcctgggagggaccaaaggcggccaggatataact
gacttcaccatgcaatttgtgtcttcctaaagagagctgtacccagagagt
cctgtgctgaatgtggactcaatccctagggctggcagaaagggaacagaa
aggtttttgagtacggctatagcctggactttcctgttgtctacaccaatg
cccaactgcctgccttagggtagtgctaagaggatctcctgtccatcagcc
aggacagtcagctctctcctttcagggccaatccccagcccttttgttgag
ccaggcctctctcacctctcctactcacttaaagcccgcctgacagaaacc
acggccacatttggttctaagaaaccctctgtcattcgctcccacattctg
atgagcaaccgcttccctatttatttatttatttgtttgtttgttttattc
attggtctaatttattcaaagggggcaagaagtagcagtgtctgtaaaaga
gcctagtttttaatagctatggaatcaattcaatttggactggtgtgctct
ctttaaatcaagtcctttaattaagactgaaaatatataagctcagattat
ttaaatgggaatatttataaatgagcaaatatcatactgttcaatggttct
gaaataaacttcactgaag

### EQUIVALENTS

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

What is claimed is:

1. A single-stranded modified oligonucleotide consisting of 10-30 linked nucleosides and having: a gap segment consisting of two to eight linked deoxynucleosides; a 5′ wing segment consisting of linked nucleosides; and a 3′ wing segment consisting of linked nucleosides; wherein the gap segment is positioned between the 5′ wing segment and the 3′ wing segment; wherein at least some nucleosides of each wing segment comprises a modified nucleotide; wherein at least 5 of the internucleoside linkages within the gap segment and the linkages connecting the gap segment to the 3′ wing segments are phosphorothioate linkages (*); and the internucleoside linkages connecting the rest of the nucleosides of both the 5′ and 3′ wing segments are phosphodiester linkages; and wherein the nucleobase sequence of the oligonucleotide consists of 10-30 contiguous nucleobases complementary to an equal length portion of SEQ ID NO: 1, or a pharmaceutically acceptable salt thereof.

2. The oligonucleotide of claim 1, wherein the nucleobase sequence of the oligonucleotide is mGmUmAmGmUmGG*T*G*G*T*/iMe-dC/*mGmGmAmGmAmU (SEQ ID NO: 43).

3. The oligonucleotide of claim 1, wherein the nucleobase sequence of the oligonucleotide is mAmGmAmUmCmCT*C*T*T*A*G*mCmAmCmUmAmC (SEQ ID NO: 3).

4. The oligonucleotide of claim 1, wherein the nucleobase sequence of the oligonucleotide is mGmAmAmGmGmAG*C*A*C*T*T*mCmAmUmCmUmG (SEQ ID NO: 6).

5. The oligonucleotide of claim 1, wherein the nucleobase sequence of the oligonucleotide is mCmCmAmAmGmGC*C*A*C*A*G*mGmUmAmUmUmU (SEQ ID NO: 7).

6. The oligonucleotide of claim 1, wherein the nucleobase sequence of the oligonucleotide is mUmCmCmAmGmCT*T*G*T*T*A*mUmUmGmAmUmU (SEQ ID NO: 10).

7. The oligonucleotide of any one of claims 1-6, wherein the compound is 18 nucleotides in length.

8. The oligonucleotide of any one of claims 1-7, further comprising a molecular species at one of the ends.

9. The oligonucleotide of claim 8, wherein the molecular species is selected from the group consisting of a spacer, a lipid, a sterol, cholesterol, stearyl, C16 alkyl chain, bile acids, cholic acid, taurocholic acid, deoxycholate, oleyl litocholic acid, oleoyl cholenic acid, glycolipids, phospholipids, sphingolipids, isoprenoids, such as steroids, vitamins, such as vitamin E, saturated fatty acids, unsaturated fatty acids, fatty acid esters, such as triglycerides, pyrenes, porphyrines, Texaphyrine, adamantane, acridines, biotin, coumarin, fluorescein, rhodamine, Texas-Red, digoxygenin, dimethoxytrityl, t-butyldimethylsilyl, t-butyldiphenylsilyl, cyanine dyes (e.g. Cy3 or Cy5), Hoechst 33258 dye, psoralen, and ibuprofen.

10. The oligonucleotide of claim 8, wherein the molecular species is connected directly to the compound through a linkage selected from the group consisting of phosphodiester, phosphorothioate, methylphosphonate, and amide linkages.

11. The oligonucleotide of claim 10, wherein the molecular species is connected indirectly to the compound through a linker.

12. The oligonucleotide of claim 11, wherein the linker is a non-nucleotidic linker selected from the group consisting of abasic residues (dSpacer), oligoethyleneglycol, such as triethyleneglycol (spacer 9) or hexaethyleneglycol (spacer 18), and alkane-diol, such as butanediol.

13. The oligonucleotide of claim 12, wherein the 3′ end of the oligonucleotide is connected to 2 consecutive linkers that are hexaethyleneglycol (spacer 18), the first hexaethyleneglycol connected to the 3′ end of the oligonucleotide, the second hexaethyleneglycol connected to the first hexaethyleneglycol and the second hexaethyleneglycol is connected to a cholesterol.

14. The oligonucleotide of claim 1, wherein the gap segment consists of six linked deoxynucleosides.

15. The oligonucleotide of claim 1, wherein all of the nucleosides of each wing segment comprise a modified nucleotide.

16. The oligonucleotide of claim 15, wherein each nucleoside of each wing segment comprises a 2′O-methyl ribonucleoside (m).

17. An oligonucleotide comprising mGmUmAmGmUmGG*T*G*G*T*/iMe-dC/*mGmGmAmGmAmU/isp18//isp18//3CholTEG/(SEQ ID NO: 43), wherein m is a 2′O methyl, and wherein * is a phosphorothioate modification.

18. An oligonucleotide comprising mAmGmAmUmCmCT*C*T*T*A*G*mCmAmCmUmAmC/isp18//isp18//3CholTEG/(SEQ ID NO: 3), wherein m is a 2′O methyl, and wherein * is a phosphorothioate modification.

19. An oligonucleotide comprising mGmAmAmGmGmAG*C*A*C*T*T*mCmAmUmCmUmG/isp18//isp18//3CholTEG/(SEQ ID NO: 6), wherein m is a 2′O methyl, and wherein * is a phosphorothioate modification.

20. An oligonucleotide comprising mCmCmAmAmGmGC*C*A*C*A*G*mGmUmAmUmUmU/isp18//isp18//3CholTEG/(SEQ ID NO: 7), wherein m is a 2′O methyl, and wherein * is a phosphorothioate modification.

21. An oligonucleotide comprising mUmCmCmAmGmCT*T*G*T*T*A*mUmUmGmAmUmU/isp18//isp18//3CholTEG/(SEQ ID NO: 10), wherein m is a 2′O methyl, and wherein * is a phosphorothioate modification.

22. A stable self-assembling nanostructure, comprising a liposomal core comprised of a lipid bilayer and having an oligonucleotide shell comprised of an antisense oligonucleotide 18 to 21 linked nucleosides in length targeted to IL-1 beta (IL1B) and linked on the oligonucleotide 3′ end to an isp18//isp18//3CholTEG, wherein the 3CholTEG interacts with lipids in the lipid bilayer such that the oligonucleotide is radially oriented and the 5′ end of the oligonucleotide faces externally from the nanostructure.

23. The nanostructure of claim 22, wherein the antisense oligonucleotide is 18 nucleotides in length.

24. The nanostructure of claim 22 or 23, wherein IL1B has a sequence of SEQ ID NO: 1.

25. The nanostructure of any one of claims 22-24, wherein the antisense oligonucleotide has 2′O methyl modifications.

26. The nanostructure of claim 25, wherein the antisense oligonucleotide is selected from the group consisting of mGmUmAmGmUmGG*T*G*G*T*/iMe-dC/*mGmGmAmGmAmU (SEQ ID NO: 43), mAmGmAmUmCmCT*C*T*T*A*G*mCmAmCmUmAmC (SEQ ID NO: 3), mGmAmAmGmGmAG*C*A*C*T*T*mCmAmUmCmUmG (SEQ ID NO: 6), mCmCmAmAmGmGC*C*A*C*A*G*mGmUmAmUmUmU (SEQ ID NO: 7), and mUmCmCmAmGmCT*T*G*T*T*A*mUmUmGmAmUmU (SEQ ID NO: 10) wherein—refers to a phosphodiester bond, * refers to a phosphorothioate bond, and m refers to a 0 methyl.

27. A method for treating a disorder, comprising:
administering to a subject having a disorder a stable self-assembling nanostructure, comprising a liposomal core comprised of a lipid bilayer and having an oligonucleotide shell comprised of an antisense oligonucleotide 18 to 21 linked nucleosides in length targeted to IL-1 beta (IL1B) and linked on the oligonucleotide 3′ end to an isp18//isp18//3CholTEG, wherein the 3CholTEG interacts with lipids in the lipid bilayer such that the oligonucleotide is radially oriented and the 5′ end of the oligonucleotide faces externally from the nanostructure. in an effective amount to treat the disorder.

28. The method of claim 27, wherein the disorder is an inflammatory disorder.

29. The method of claim 27, wherein the disorder is selected from atopic dermatitis, Epidermolysis bullosa, uveitis, Gout, Polymyalgia rheumatica, Osteoarthritis, Systemic-onset juvenile idiopathic arthritis, Schnitzler syndrome, Familial Mediterranean fever, Cryopyrin-associated periodic syndrome (CAPS), Hyper-IgD syndrome (HIDS), TNF receptor-associated periodic syndrome (TRAPS), Type 2 diabetes, Proliferative diabetic retinopathy, Wet age-related macular degeneration, Chronic obstructive pulmonary disease, Type 1 diabetes, Urticarial vasculitis, Pyoderma gangrenosum, Dry eye syndrome, Acne vulgaris, as well as improve arterial function in atherosclerosis in type 2 diabetes and reduce cardiovascular risk in type 2 diabetes.

30. A method for reducing expression levels of IL1B in vivo, comprising:
administering to a subject a pharmaceutical composition comprising a stable self-assembling nanostructure, comprising a liposomal core comprised of a lipid bilayer and having an oligonucleotide shell comprised of an antisense oligonucleotide 18 to 21 linked nucleosides in length targeted to IL-1 beta (IL1B) and linked on the oligonucleotide 3′ end to an isp18//isp18//3CholTEG, wherein the 3CholTEG interacts with lipids in the lipid bilayer such that the oligonucleotide is radially oriented and the 5′ end of the oligonucleotide faces externally from the nanostructure in an effective amount to reduce IL1B levels in vivo.

31. The method of claim 30, wherein the subject is a mammal.

32. The method of claim 30, wherein the subject is human.

33. A method for reducing expression levels of IL1B in vitro, comprising:
contacting a cell with a stable self-assembling nanostructure, comprising a liposomal core comprised of a lipid bilayer and having an oligonucleotide shell comprised of an antisense oligonucleotide 18 to 21 linked nucleosides in length targeted to IL-1 beta (IL1B) and linked on the oligonucleotide 3′ end to an isp18//isp18//3CholTEG, wherein the 3CholTEG interacts with lipids in the lipid bilayer such that the oligonucleotide is radially oriented and the 5′ end of the oligonucleotide faces externally from the nanostructure in an effective amount to reduce IL1B levels in vitro.

34. The method of claim 33, wherein the cell is a keratinocyte.


SEQUENCE LISTING (145 sequences)

SEQ ID NO: 1 - DNA, 1498 bp, Homo sapiens
accaaacctcttcgaggcacaaggcacaacaggctgctctgggattctcttcagccaatcttcattgctcaagtgtctgaagcagccatggcagaagtacctgagctcgccagtgaaatgatggcttattacagtggcaatgaggatgacttgttctttgaagctgatggccctaaacagatgaagtgctccttccaggacctggacctctgccctctggatggcggcatccagctacgaatctccgaccaccactacagcaagggcttcaggcaggccgcgtcagttgttgtggccatggacaagctgaggaagatgctggttccctgcccacagaccttccaggagaatgacctgagcaccttctttcccttcatctttgaagaagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaaagagagctgtacccagagagtcctgtgctgaatgtggactcaatccctagggctggcagaaagggaacagaaaggtttttgagtacggctatagcctggactttcctgttgtctacaccaatgcccaactgcctgccttagggtagtgctaagaggatctcctgtccatcagccaggacagtcagctctctcctttcagggccaatccccagcccttttgttgagccaggcctctctcacctctcctactcacttaaagcccgcctgacagaaaccacggccacatttggttctaagaaaccctctgtcattcgctcccacattctgatgagcaaccgcttccctatttatttatttatttgtttgtttgttttattcattggtctaatttattcaaagggggcaagaagtagcagtgtctgtaaaagagcctagtttttaatagctatggaatcaattcaatttggactggtgtgctctctttaaatcaagtcctttaattaagactgaaaatatataagctcagattatttaaatgggaatatttataaatgagcaaatatcatactgttcaatggttctgaaataaacttcactgaag

SEQ ID NO: 2 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
ggucggagattcguagcu

SEQ ID NO: 3 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
agaucctcttagcacuac

SEQ ID NO: 4 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
guaguggtggtcggagau

SEQ ID NO: 5 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
gcauggtgaagtcaguua

SEQ ID NO: 6 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
gaaggagcacttcaucug

SEQ ID NO: 7 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
ccaaggccacagguauuu

SEQ ID NO: 8 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
ccaaaugtggccgugguu

SEQ ID NO: 9 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
gaaggagcactgcggaga

SEQ ID NO: 10 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
uccagcttgttauugauu

SEQ ID NO: 11 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
ugcauggtgaagucaguu

SEQ ID NO: 12 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
aucccatgtgtcgaagaa

SEQ ID NO: 13 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
guggucggagatucguag

SEQ ID NO: 14 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
uguaguggtggtcggaga

SEQ ID NO: 15 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
cacaacaactgacgcggc

SEQ ID NO: 16 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
aaucugagcttauauauu

SEQ ID NO: 17 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
guuuuctgcttgagaggu

SEQ ID NO: 18 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
cagcuugttattgauuuc

SEQ ID NO: 19 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
gcuaactaccttcuucaa

SEQ ID NO: 20 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
ccaagcttttttgcugug

SEQ ID NO: 21 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
aauaauctgagcuuauau

SEQ ID NO: 22 - DNA, 18 bp, Artificial Sequence
  Synthetic polynucleotide
  misc_feature (1)..(4): may be a 2′-O-methylated base
  misc_feature (5)..(5): n is a  2-O-methyl7-deaza-deoxyguanosine
  misc_feature (6)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
ggcgngctttaagugagu

SEQ ID NO: 23 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
uucagacacctguguaaa

SEQ ID NO: 24 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(13): may be a 2′-O-methylated base
  misc_feature (14)..(14): n is a 2-O-methyl7-deaza-deoxyguanosine
  misc_feature (15)..(18): may be a 2′-O-methylated base
  misc_feature (18)..(18): may be modified by two internal hexaethylene gylcol spacers, and a 3′ tri(ethyleneglycol) bound to a cholesterylester
ucaucagaatgtgngagc

SEQ ID NO: 25 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
uccacugacctguaauaa

SEQ ID NO: 26 - DNA, 18 bp, Artificial Sequence
  Synthetic polynucleotide
  misc_feature (1)..(6): may be a 2′-O-methylated base
  misc_feature (7)..(8): may be a phosphorothioate bond
  misc_feature (9)..(9): n is a phosphorothioated 7-deaza-deoxyguanosine
  misc_feature (10)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
auaaauagngaagcgguu

SEQ ID NO: 27 - DNA, 18 bp, Artificial Sequence
  Synthetic polynucleotide
  misc_feature (1)..(3): may be a 2′-O-methylated base
  misc_feature (4)..(4): n is a 2-O-methyl7-deaza-deoxyguanosine
  misc_feature (5)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
acgngcatgtttucugcu

SEQ ID NO: 28 - DNA, 18 bp, Artificial Sequence
  Synthetic polynucleotide
  misc_feature (1)..(4): may be a 2′-O-methylated base
  misc_feature (5)..(5): n is a 2-O-methyl7-deaza-deoxyguanosine
  misc_feature (6)..(6): may be a 2′-O-methylated base
  misc_feature (7)..(12): may be a phosphorothioate bond
  misc_feature (13)..(15): may be a 2′-O-methylated base
  misc_feature (16)..(16): n is a 2-O-methyl7-deaza-deoxyguanosine
  misc_feature (17)..(18): may be a 2′-O-methylated base
  misc_feature (18)..(18): may be modified by two internal hexaethylene gylcol spacers, and a 3′ tri(ethyleneglycol) bound to a cholesterylester
uuugngtaatttuugnga

SEQ ID NO: 29 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
ucauugccactguaauaa

SEQ ID NO: 30 - DNA, 18 bp, Artificial Sequence
  Synthetic polynucleotide
  misc_feature (1)..(6): may be a 2′-O-methylated base
  misc_feature (7)..(9): may be a phosphorothioate bond
  misc_feature (10)..(10): n is a phosphorothioated 7-deaza-deoxyguanosine
  misc_feature (11)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
ucuucuttgngtaauuuu

SEQ ID NO: 31 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
agauaggttctgaaaugu

SEQ ID NO: 32 - DNA, 18 bp, Artificial Sequence
  Synthetic polynucleotide
  misc_feature (1)..(6): may be a 2′-O-methylated base
  misc_feature (7)..(10): may be a phosphorothioate bond
  misc_feature (11)..(11): n is a  phosphorothioated 7-deaza- deoxyguanosine
  misc_feature (12)..(13): may be a phosphorothioate bond
  misc_feature (14)..(18): may be a 2′-O-methylated base
  misc_feature (18)..(18): may be modified by two internal hexaethylene gylcol spacers, and a 3′ tri(ethyleneglycol) bound to a cholesterylester
ucagaatgtgngagcgaa

SEQ ID NO: 33 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
agaacaccacttguugcu

SEQ ID NO: 34 - DNA, 18 bp, Artificial Sequence
  Synthetic polynucleotide
  misc_feature (1)..(6): may be a 2′-O-methylated base
  misc_feature (7)..(7): n is a phosphorothioated 7-deaza-deoxyguanosine
  misc_feature (8)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
aaauagngaagcgguugc

SEQ ID NO: 35 - DNA, 18 bp, Artificial Sequence
  Synthetic polynucleotide
  misc_feature (1)..(6): may be a 2′-O-methylated base
  misc_feature (7)..(9): may be a phosphorothioate bond
  misc_feature (10)..(10): n is a phosphorothioated 7-deaza-deoxyguanosine
  misc_feature (11)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
cagaaugtgngagcgaau

SEQ ID NO: 36 - DNA, 18 bp, Artificial Sequence
  Synthetic polynucleotide
  misc_feature (1)..(4): may be a 2′-O-methylated base
  misc_feature (5)..(5): n is a 2-O-methyl7-deaza-deoxyguanosine
  misc_feature (6)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
agagngtttcttagaacc

SEQ ID NO: 37 - DNA, 18 bp, Artificial Sequence
  Synthetic polynucleotide
  misc_feature (1)..(6): may be a 2′-O-methylated base
  misc_feature (7)..(7): may be a phosphorothioate bond
  misc_feature (8)..(8): n is a phosphorothioated 7-deaza-deoxyguanosine
  misc_feature (9)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
uaaauagngaagcgguug

SEQ ID NO: 38 - DNA, 18 bp, Artificial Sequence
  Synthetic polynucleotide
  misc_feature (1)..(3): may be a 2′-O-methylated base
  misc_feature (4)..(4): n is a 2-O-methyl7-deaza-deoxyguanosine
  misc_feature (5)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
gagnguttcttagaacca

SEQ ID NO: 39 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
ucauugccactguaauaa

SEQ ID NO: 40 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
uucacutacctccagcug

SEQ ID NO: 41 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
aucuacactctgcagcga

SEQ ID NO: 42 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
aucuacactctccagcug

SEQ ID NO: 43 - DNA, 18 bp, Artificial Sequence
  Synthetic polynucleotide
  misc_feature (1)..(6): may be a 2′-O-methylated base
  misc_feature (7)..(11): may be a phosphorothioate bond
  misc_feature (12)..(12): n is a phosphorothioated iMe-dC
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
guaguggtggtnggagau

SEQ ID NO: 44 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
ugucgaagaagauagguu

SEQ ID NO: 45 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
gugucgaagaagauaggu

SEQ ID NO: 46 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
uuaucccatgtgucgaag

SEQ ID NO: 47 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
guuaucccatgtgucgaa

SEQ ID NO: 48 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
cguuaucccatgugucga

SEQ ID NO: 49 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
ucguuatcccatgugucg

SEQ ID NO: 50 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
cucguuatcccauguguc

SEQ ID NO: 51 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
ccucgutatcccaugugu

SEQ ID NO: 52 - DNA, 18 bp, Artificial Sequence
  Synthetic polynucleotide
  misc_feature (1)..(6): may be an e-modified base
  misc_feature (7)..(12): may be a phosphorothioate bond
  misc_feature (13)..(18): may b an e-modified base
  misc_feature (18)..(18): may be modified by two internal hexaethylene gylcol spacers, and a 3′ tri(ethyleneglycol) bound to a cholesterylester
gccucgttatcccaugug

SEQ ID NO: 53 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
gccucgttatcccaugug

SEQ ID NO: 54 - DNA, 18 bp, Artificial Sequence
  Synthetic polynucleotide
  misc_feature (1)..(3): may be a 2′-O-methylated base
  misc_feature (4)..(15): may be a phosphorothioate bond
  misc_feature (16)..(18): may be a 2′-O-methylated base
  misc_feature (18)..(18): may be modified by two internal hexaethylene gylcol spacers, and a 3′ tri(ethyleneglycol) bound to a cholesterylester
gcctcgttatcccatgug

SEQ ID NO: 55 - DNA, 18 bp, Artificial Sequence
  Synthetic polynucleotide
  misc_feature (1)..(5): may be a 2′-O-methylated base
  misc_feature (6)..(11): may be a phosphorothioate bond
  misc_feature (12)..(18): may be a 2′-O-methylated base
  misc_feature (18)..(18): may be modified by two internal hexaethylene gylcol spacers, and a 3′ tri(ethyleneglycol) bound to a cholesterylester
gccucgttatcccaugug

SEQ ID NO: 56 - DNA, 18 bp, Artificial Sequence
  Synthetic polynucleotide
  misc_feature (1)..(5): may be a 2′-O-methylated base
  misc_feature (6)..(13): may be a phosphorothioate bond
  misc_feature (14)..(18): may be a 2′-O-methylated base
  misc_feature (18)..(18): may be modified by two internal hexaethylene gylcol spacers, and a 3′ tri(ethyleneglycol) bound to a cholesterylester
gccucgttatcccaugug

SEQ ID NO: 57 - DNA, 18 bp, Artificial Sequence
  Synthetic polynucleotide
  misc_feature (1)..(7): may be a 2′-O-methylated base
  misc_feature (8)..(13): may be a phosphorothioate bond
  misc_feature (14)..(18): may be a 2′-O-methylated base
  misc_feature (18)..(18): may be modified by two internal hexaethylene gylcol spacers, and a 3′ tri(ethyleneglycol) bound to a cholesterylester
gccucgutatcccaugug

SEQ ID NO: 58 - DNA, 18 bp, Artificial Sequence
  Synthetic polynucleotide
  misc_feature (1)..(9): may be a 2′-O-methylated base
  misc_feature (10)..(15): may be a phosphorothioate bond
  misc_feature (16)..(18): may be a 2′-O-methylated base
  misc_feature (18)..(18): may be modified by two internal hexaethylene gylcol spacers, and a 3′ tri(ethyleneglycol) bound to a cholesterylester
gccucguuatcccatgug

SEQ ID NO: 59 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): modified by a 3′ tri(ethyleneglycol) bound to a cholesterylester
gccucgttatcccaugug

SEQ ID NO: 60 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by polyethyleneglycol with an average molecular weight of 2,500 dalton, and a 3′ tri (ethyleneglycol) bound to a cholesterylester
gccucgttatcccaugug

SEQ ID NO: 61 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
agccucgttatcccaugu

SEQ ID NO: 62 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
aagccucgttatcccaug

SEQ ID NO: 63 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
uaagcctcgttaucccau

SEQ ID NO: 64 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
auaagcctcgttauccca

SEQ ID NO: 65 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
uggagagctttcaguuca

SEQ ID NO: 66 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
guggagagctttcaguuc

SEQ ID NO: 67 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
gguggagagcttucaguu

SEQ ID NO: 68 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
agguggagagctuucagu

SEQ ID NO: 69 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
gagguggagagcuuucag

SEQ ID NO: 70 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
ggagguggagagcuuuca

SEQ ID NO: 71 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
uggaggtggagagcuuuc

SEQ ID NO: 72 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
cuggaggtggagagcuuu

SEQ ID NO: 73 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
ccuggaggtggagagcuu

SEQ ID NO: 74 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
cccuggaggtggagagcu

SEQ ID NO: 75 - DNA, 18 bp, Artificial Sequence
  Synthetic polynucleotide
  misc_feature (1)..(3): may be a 2′-O-methylated base
  misc_feature (4)..(15): may be a phosphorothioate bond
  misc_feature (16)..(18): may be a 2′-O-methylated base
  misc_feature (18)..(18): may be modified by two internal hexaethylene gylcol spacers, and a 3′ tri(ethyleneglycol) bound to a cholesterylester
ccctggaggtggagagcu

SEQ ID NO: 76 - DNA, 18 bp, Artificial Sequence
  Synthetic polynucleotide
  misc_feature (1)..(3): may be a 2′-O-methylated base
  misc_feature (4)..(9): may be a phosphorothioate bond
  misc_feature (10)..(18): may be a 2′-O-methylated base
  misc_feature (18)..(18): may be modified by two internal hexaethylene gylcol spacers, and a 3′ tri(ethyleneglycol) bound to a cholesterylester
ccctggagguggagagcu

SEQ ID NO: 77 - DNA, 18 bp, Artificial Sequence
  Synthetic polynucleotide
  misc_feature (1)..(5): may be a 2′-O-methylated base
  misc_feature (6)..(11): may be a phosphorothioate bond
  misc_feature (12)..(18): may be a 2′-O-methylated base
  misc_feature (18)..(18): may be modified by two internal hexaethylene gylcol spacers, and a 3′ tri(ethyleneglycol) bound to a cholesterylester
cccuggaggtggagagcu

SEQ ID NO: 78 - DNA, 18 bp, Artificial Sequence
  Synthetic polynucleotide
  misc_feature (1)..(5): may be a 2′-O-methylated base
  misc_feature (6)..(13): may be a phosphorothioate bond
  misc_feature (14)..(18): may be a 2′-O-methylated base
  misc_feature (18)..(18): may be modified by two internal hexaethylene gylcol spacers, and a 3′ tri(ethyleneglycol) bound to a cholesterylester
cccuggaggtggagagcu

SEQ ID NO: 79 - DNA, 18 bp, Artificial Sequence
  Synthetic polynucleotide
  misc_feature (1)..(7): may be a 2′-O-methylated base
  misc_feature (8)..(13): may be a phosphorothioate bond
  misc_feature (14)..(18): may be a 2′-O-methylated base
  misc_feature (18)..(18): may be modified by two internal hexaethylene gylcol spacers, and a 3′ tri(ethyleneglycol) bound to a cholesterylester
cccuggaggtggagagcu

SEQ ID NO: 80 - DNA, 18 bp, Artificial Sequence
  Synthetic polynucleotide
  misc_feature (1)..(9): may be a 2′-O-methylated base
  misc_feature (10)..(15): may be a phosphorothioate bond
  misc_feature (16)..(18): may be a 2′-O-methylated base
  misc_feature (18)..(18): may be modified by two internal hexaethylene gylcol spacers, and a 3′ tri(ethyleneglycol) bound to a cholesterylester
cccuggaggtggagagcu

SEQ ID NO: 81 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
ucccuggaggtggagagc

SEQ ID NO: 82 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
guauuutgtcatuacuuu

SEQ ID NO: 83 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
gguauuttgtcauuacuu

SEQ ID NO: 84 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
cagguattttgtcauuac

SEQ ID NO: 85 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
acagguattttgucauua

SEQ ID NO: 86 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
cacaggtattttgucauu

SEQ ID NO: 87 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
ccacaggtatttugucau

SEQ ID NO: 88 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
gccacaggtattuuguca

SEQ ID NO: 89 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
ggccacaggtatuuuguc

SEQ ID NO: 90 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
aggccacaggtauuuugu

SEQ ID NO: 91 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
aaggccacaggtauuuug

SEQ ID NO: 92 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
caaggccacagguauuuu

SEQ ID NO: 93 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
cccaaggccacagguauu

SEQ ID NO: 94 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
uucuuugggtaauuuuug

SEQ ID NO: 95 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
cuucuutgggtaauuuuu

SEQ ID NO: 96 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
ucuucuttgggtaauuuu

SEQ ID NO: 97 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
uucuuctttggguaauuu

SEQ ID NO: 98 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
uuccagcttgttauugau

SEQ ID NO: 99 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
aauuccagcttguuauug

SEQ ID NO: 100 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
caaauuccagctuguuau

SEQ ID NO: 101 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
guaccagttggggaacug

SEQ ID NO: 102 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
auguaccagttggggaac

SEQ ID NO: 103 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
ugauguaccagtugggga

SEQ ID NO: 104 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
aggugctgatgtaccagu

SEQ ID NO: 105 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
gaggugctgatguaccag

SEQ ID NO: 106 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
agaggugctgatguacca

SEQ ID NO: 107 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
gagaggtgctgauguacc

SEQ ID NO: 108 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
ugagaggtgctgauguac

SEQ ID NO: 109 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
uugagaggtgctgaugua

SEQ ID NO: 110 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
cuugagaggtgcugaugu

SEQ ID NO: 111 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
gcuugagaggtgcugaug

SEQ ID NO: 112 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
ugcuugagaggtgcugau

SEQ ID NO: 113 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
cugcuugagaggugcuga

SEQ ID NO: 114 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
ucugcutgagaggugcug

SEQ ID NO: 115 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
cgggcatgttttcugcuu

SEQ ID NO: 116 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
gacgggcatgttuucugc

SEQ ID NO: 117 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
aagacgggcatguuuucu

SEQ ID NO: 118 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
gaagacgggcatguuuuc

SEQ ID NO: 119 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
aauugcatggtgaaguca

SEQ ID NO: 120 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
aaauugcatggtgaaguc

SEQ ID NO: 121 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
caaauugcatggugaagu

SEQ ID NO: 122 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
acaaautgcatggugaag

SEQ ID NO: 123 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
cacaaattgcatggugaa

SEQ ID NO: 124 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
uugggcattggtguagac

SEQ ID NO: 125 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
guugggcattgguguaga

SEQ ID NO: 126 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
aguugggcattgguguag

SEQ ID NO: 127 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
caguugggcattggugua

SEQ ID NO: 128 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
gcaguugggcatuggugu

SEQ ID NO: 129 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
ggcagutgggcauuggug

SEQ ID NO: 130 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
aggcagttgggcauuggu

SEQ ID NO: 131 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
caggcagttgggcauugg

SEQ ID NO: 132 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
cagagggtttctuagaac

SEQ ID NO: 133 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
gacagagggtttcuuaga

SEQ ID NO: 134 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
aaugacagaggguuucuu

SEQ ID NO: 135 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
aauaaatagggaagcggu

SEQ ID NO: 136 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
uaaauaaatagggaagcg

SEQ ID NO: 137 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
aauaaataaatagggaag

SEQ ID NO: 138 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
gagcuuatatatuuucag

SEQ ID NO: 139 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
cugagcttatatauuuuc

SEQ ID NO: 140 - DNA, 18 bp, Artificial Sequence
  Synthetic polynucleotide
  misc_feature (1)..(6): may be a 2′-O-methylated base
  misc_feature (7)..(12): may be a phosphorothioate bond
  misc_feature (13)..(18): may be a 2′-O-methylated base
  misc_feature (18)..(18): may be modified by two internal hexaethylene gylcol spacers, and a 3′ tri(ethyleneglycol) bound to a cholesterylester
guaguggtggtcggagau

SEQ ID NO: 141 - DNA, 18 bp, Artificial Sequence
  Synthetic polynucleotide
  misc_feature (1)..(5): may be a 2′-O-methylated base
  misc_feature (6)..(11): may be a phosphorothioate bond
  misc_feature (12)..(18): may be a 2′-O-methylated base
  misc_feature (18)..(18): may be modified by two internal hexaethylene gylcol spacers, and a 3′ tri(ethyleneglycol) bound to a cholesterylester
guaguggtggtcggagau

SEQ ID NO: 142 - DNA, 23 bp, Artificial Sequence
  Synthetic polynucleotide
caaggtcatccatgacaactttg

SEQ ID NO: 143 - DNA, 19 bp, Artificial Sequence
  Synthetic polynucleotide
gggccatccacagtcttct

SEQ ID NO: 144 - DNA, 25 bp, Artificial Sequence
  Synthetic polynucleotide
accacagtccatgccatcactgcca

SEQ ID NO: 145 - DNA, 1421 bp, Homo sapiens
gcctcaagaccttgggctgggactggctgagcctggcgggaggcggggtccgagtcaccgcctgccgccgcgcccccggtttctataaattgagcccgcagcctcccgcttcgctctctgctcctcctgttcgacagtcagccgcatcttcttttgcgtcgccagccgagccacatcgctcagacaccatggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgacaacgaatttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaaccagttaaaaaaaaaaaaaaaaaaaaa